(19) United States
(12) Patent Application Publication
(10) Pub. No.: US 2024/0000335 A1
(43) Pub. Date: Jan. 4, 2024

(54) IMPEDANCE BASED WOUND HEALING MONITOR

(71) Applicant: 3M INNOVATIVE PROPERTIES COMPANY, St. Paul, MN (US)

(72) Inventors: Stephen M. Kennedy, St. Paul, MN (US); Jeffrey P. Kalish, St. Paul, MN (US); Sean M. Sweetnam, Minneapolis, MN (US); Michael D. Benson, Mendota Heights, MN (US); Jana Ninkovic, St. Paul, MN (US); Jason W. Bjork, Cottage Grove, MN (US); Amy K. McNulty, Stillwater, MN (US)

(21) Appl. No.: 18/039,692

(22) PCT Filed: Nov. 10, 2021
(86) PCT No.: PCT/IB2021/060426

§ 371 Date: May 31, 2023

Related U.S. Application Data
(60) Provisional application No. 63/120,427, filed on Dec. 2, 2020.

(51) Int. Cl. A61B 5/0531; A61B 5/00

(52) U.S. Cl. CPC A61B 5/0531; A61B 5/445; A61B 5/683; A61B 2562/164

(57) ABSTRACT

A method includes applying an electrical signal to a tissue and measuring an impedance of the tissue based on the applied electrical signal. The method further includes determining information indicative of a stage of wound healing based on the impedance and outputting information indicative of the stage of wound healing.

### TECHNICAL FIELD

The disclosure relates to wound monitoring.

### BACKGROUND

Smart wound dressings and other wearable sensors are used to monitor healing. The ability to monitor healing may lead to improved healthcare and improved patient outcomes. Monitoring healing may be used to determine whether a current treatment program is effective, or whether changes to a current treatment program should be made.

### SUMMARY

In general, the disclosure describes example techniques for determining and/or tracking physiological measures of wound healing using electrical signals, such as, the impedance at one or more locations at a wound site.

One or more pairs of electrodes may apply an electrical signal, or signals, to a tissue site, or a location within a tissue site, and processing circuitry may determine an impedance of the wounded tissue at the location based on the applied electrical signal or signals. The tissue site may correspond to a wound, e.g., tissue having damage. In some examples, the processing circuitry determines a baseline impedance (e.g., impedance at unwounded locations proximate to the wound, or pre-stored impedance information such as expected impedance based on an average of patients and patients of particular demographic profiles). The processing circuitry may determine information indicative of tissue characteristics based on the impedance of the wounded tissue, both the baseline impedance and the impedance of the wounded tissue, or the impedance of the wounded and unwounded tissues at different times. The information indicative of tissue characteristics may include a stage of healing. For example, the processing circuitry may determine a stage of healing based on the impedance of wounded tissue, and in some examples whether a wound is chronic, e.g., not healing. That is the wound being chronic is an example of remaining in a particular stage of healing for a prolonged period of time (i.e., indicative of not healing).

The techniques disclosed may provide simplified, low-cost techniques capable of providing real-time, clinically actionable output related to healing, or lack thereof. For instance, utilizing impedance may provide a more accurate measure of the patient healing as compared to other techniques. Continuous monitoring and capture of real-time wound healing data may lead to improved healthcare and improved patient outcomes.

In one example, this disclosure describes a method that includes applying an electrical signal to a tissue, measuring an impedance of the tissue based on the applied electrical signal;

determining information indicative of a stage of wound healing based on the impedance, and outputting information indicative of the stage of wound healing.

In another example, this disclosure describes a system includes a first device configured to apply an electrical signal to a tissue; and a second device configured to measure an impedance of the tissue based on the applied electrical signal, to determine information indicative of a stage of wound healing based on the impedance, and to output information indicative of the stage of wound healing.

In another example, this disclosure describes a dressing includes electrical contacts configured to be coupled to wounded tissue and to unwounded tissue proximate to wounded tissue, a first device configured to apply an electrical signal through the wounded tissue via the electrical contacts at two or more predetermined time intervals, and a second device configured to: measure an impedance of the first tissue based on the applied electrical signal at each of the two or more predetermined time intervals, determine a resistance and a reactance at each of the two or more predetermined time intervals based on the measured impedances, determine a stage of healing based on the determined resistances and reactances at each of the two or more predetermined time intervals; and output the stage of healing.

The details of one or more examples are set forth in the accompanying drawings and the description below. Other features, objects, and advantages will be apparent from the description and drawings, and from the claims.

### BRIEF DESCRIPTION OF DRAWINGS

FIG. 1 is an illustration depicting an example wound monitoring system, in accordance with the techniques described in this disclosure.

FIG. 2A is a conceptual diagram illustrating an aerial view an example tissue site, in accordance with one or more techniques of this disclosure.

FIG. 2B is a conceptual diagram illustrating a side-view of the example tissue site of FIG. 2A, in accordance with one or more techniques of this disclosure.

FIG. 3A is a conceptual diagram illustrating an aerial view an example tissue site, in accordance with one or more techniques of this disclosure.

FIG. 3B is a conceptual diagram illustrating a side-view of the example tissue site of FIG. 3A, in accordance with one or more techniques of this disclosure.

FIG. 4 is an illustration depicting an example wound monitoring system, in accordance with the techniques described in this disclosure.

FIG. 5A is a plot of example impedance magnitude as a function of signal frequency for a tissue site at a plurality of times after a wound at the tissue site, in accordance with the techniques described in this disclosure.

FIG. 5B is a plot of example impedance phase angle as a function of signal frequency for a tissue site at a plurality of times after a wound at the tissue site, in accordance with the techniques described in this disclosure.

FIGS. 6A-6E are conceptual cross-sectional schematic diagrams of a wound site at various stages of healing, in accordance with the techniques described in this disclosure.

FIG. 7A is a plot of example complex impedances of a tissue site at a predetermined frequency measured at a plurality of times after a wound occurs at the tissue site, in accordance with the techniques described in this disclosure.

FIG. 7B is a plot of example complex impedances of a plurality of tissue types at a predetermined frequency measured at a plurality of times after a wound occurs at the tissue site, in accordance with the techniques described in this disclosure.

FIG. 8 is a flowchart of an example method of monitoring tissue characteristics, in accordance with one or more techniques of this disclosure.

FIG. 9A is a plot of example wound bed resistance measured at a predetermined frequency as a function of time for a tissue site, in accordance with the techniques described in this disclosure.

FIG. 9B is a plot of example granular tissue thicknesses as a function of wound bed resistance measured at a predetermined frequency for a tissue site at a plurality of times, in accordance with the techniques described in this disclosure.

FIG. 10 is a plot of example granular tissue thicknesses as a function of wound bed resistance measured at a predetermined frequency for a tissue site at a plurality of times overlaid with an exponential curve fit of the granular tissue thickness, in accordance with the techniques described in this disclosure.

FIG. 11 is a flowchart of an example method of monitoring tissue characteristics, in accordance with one or more techniques of this disclosure.

FIG. 12A is a plot of example reactances measured at a predetermined frequency as a function of time for a tissue site, in accordance with the techniques described in this disclosure.

FIG. 12B is a plot of example impedance phase angles measured at a predetermined frequency as a function of time for a tissue site, in accordance with the techniques described in this disclosure.

FIG. 13 is a flowchart of an example method of monitoring tissue characteristics, in accordance with one or more techniques of this disclosure.

FIG. 14A is a plot of example reactance ratios and/or impedance phase angle ratios measured at a predetermined frequency as a function of time, in accordance with the techniques described in this disclosure.

FIG. 14B is a plot of an example corresponding epithelial coverage based on the reactance ratios and/or impedance phase angles of FIG. 14A as a function of time, in accordance with the techniques described in this disclosure.

### DETAILED DESCRIPTION

In examples, the disclosure describes a method and system for tracking quantitative metrics of wound healing by measuring the impedance of tissues in and proximate to wound beds. In general, a wound may include tissue having damage, bruised tissue, burned tissue, scraped tissue, tissue having a rash, deeper wounds extending within subcutaneous tissue, incisional and/or surgical wounds, and the like.

Tissue impedance may undergo characteristic changes during wound healing, and this change versus time may be used to quantitatively track wound healing. For example, unwounded tissues may exhibit capacitive charging over a certain range of frequencies. That is, unwounded tissues comprise epithelial layers with high cell densities and tight cell-cell junctions which serve as a barrier upon which charge will accumulate. As such, the top layers of tissues act as a parallel plate capacitor. This capacitance will manifest as a complex impedance with a phase angle θ between −90° and 0° over a range of frequencies associated with the charging timescales of the epithelial layer. However, when wounded, these tissue layers can be damaged or removed, reducing the tissue's inherent electrical capacitance. This loss of capacitance will manifest as a shorting of the complex impedance's phase angle towards 0° over this range in frequencies. In other words, the complex impedance of damaged or removed tissue may no longer be complex and may be substantially resistive having a resistance R.

As the wound begins to heal, the tissue may progress through a series of stages, and the electrical characteristics of the wounded tissue may correspondingly progress through a series of stages that are distinguishable from each other. For example, tissue that is damaged or removed may initially be in an inflammation stage including hemostasis and inflammatory response. During this first stage (e.g., inflammation stage), the electrical impedance of the tissue may be substantially resistive with substantially little reactance. As the wound heals through granulation tissue production, the wound progresses to a second, or proliferation, stage. During the proliferation stage (e.g., a granulation stage), the electrical impedance of the tissue may still be substantially resistive with substantially little reactance, but the resistance may be reduced relative to the first stage, e.g., the granulation tissue may be more conductive than the native tissue. The electrical impedance may still have substantially little reactance because the granulation tissue may be disorganized and lack structures that facilitate electrical charging.

During a third, or remodeling stage when the wound re-epithelializes, tightly joined epithelial layers are gradually restored, by development of an epithelial monolayer and then a thickening of this monolayer through epithelial proliferation, and then with a de-nucleation of cells contained in the epithelium's topmost layer. The nascent epithelial layer may provide an electrical boundary that may facilitate electrical charging, and the electrical impedance during remodeling/re-epithelialization may exhibit an increasing negative reactance (e.g., capacitance characterized by the phase of an electrical voltage and/or current passing through the tissue lagging the input electrical signal voltage and/or current) as the epithelial layers are gradually restored. During a fourth, or healed stage, the electrical impedance of the substantially re-epithelialized and healed wound may be restored to the original impedance and/or an impedance substantially similar to nearby unwounded tissue.

As such, monitoring impedance versus time may indicate the amount of healing experienced in the wound bed and provide a technique for tracking tissue characteristics via electrical response of the wounded tissue, and which may be indicative of physiologically relevant and quantitative metrics of healing. For example, monitoring impedance versus time of a wound may indicate a stage of healing at the time of a measurement of the impedance. Monitoring impedance versus time may further indicate if a wound may be considered chronic, e.g., the wound is not progressing through stages of healing. For example, if the impedance of wound does not progress from a first stage having a first electrical resistance to a second stage having a second resistance less than the first resistance, e.g., indicating increasing growth of granulation tissue, in a certain period of time such as 1-4 weeks, the wound may be characterized as chronic or stalled requiring further inspection and/or treatment to facilitate healing. Impedance may be measured by applying electrical signals into the tissue and measuring the signals' voltages and currents.

In general, tracking impedance may require relatively few components and can be measured with relatively low-cost components. The increasing adoption of telemedicine healthcare paradigms contribute to the need for systems that enable remote monitoring. Accordingly, this disclosure provides a way to determine how much a wound has healed using relatively few low-cost components that may be integrated into a system that can be incorporated into a remote monitoring system.

The ability to monitor wound healing without removing a dressing material (which can significantly disrupt the wound bed) may be a powerful tool, particularly if impedance data is indicative of quantitative, physiological metrics of healing. For example, simply knowing if (and to what degree) a wound is healing or not will inform clinicians or the patient/customer if intervention is required or not (e.g., changing the dressing, cleaning the wound, administering therapeutics to the wound). Monitoring of wound healing through impedance may reduce the need for frequent dressing changes and may provide an additional and more detailed wound progression history. For example, with each dressing removal the tissue gets traumatized and healing gets delayed. By monitoring wounds, the frequency of dressing changes may be reduced, thereby reducing tissue trauma and pain and improving healing over the duration of treatment. Chronic wounds pose a major threat to public health and healthcare economies. Chronic wounds are also most prevalent in rapidly expanding populations of elderly, obese, and diabetic patients.

In examples, measurement of impedance correlates strongly to several physiological metrics of healing associated with inflammation, granulation, and the continuity, size, and degree of maturation of an epithelial layer regenerated over the wound. Measurements of the impedance may detect delays in healing and may detect differences in healing rate/quality between tissues exhibiting different degrees of healing, e.g., between different humans and/or different animals. Measurement of the impedance of a wound site and/or location may be robust across multiple wound sites and across different animals. The techniques disclosed in this disclosure may be implemented with non-complex, low-cost electronics which could be integrated directly into “smart” wound dressings capable of providing real-time, clinically actionable readouts of healing (or a lack of healing, e.g., when detecting chronic wounds).

In examples, one or more pairs of electrodes may apply one or more electrical signals to a wound site, or to a location within a wound site, and processing circuitry may determine an impedance of the wounded tissue at the location based on the applied electrical signal or signals. Additionally, one or more pairs of electrodes may apply one or more electrical signals to an unwounded location (e.g., proximate to the wound site but not limited to being proximate) and determine a baseline impedance at the unwounded location based on the applied electrical signal or signals. In some examples, an electrical signal apparatus may apply one or more electrical signals at a wound site and/or at an unwounded location via any suitable means, including but not limited to non-contact techniques such as inductive coupling or capacitive coupling. In some examples, the processing circuitry may determine a baseline impedance based on a stored impedance of the wounded site before wounding, or stored impedance information such as expected impedance based on an average of patients, patients of particular demographics, and/or the anatomical location of the wound. Information indicative of wound healing and/or a healing stage may be determined based on the impedance of the wounded tissue or both the baseline impedance and the impedance of the wounded tissue.

In some examples, information indicative of wound healing and/or a wound healing stage may be determined based on a single-frequency impedance, e.g., the impedance measured via application of an electrical signal comprising a predetermined, and/or substantially single, frequency. In other examples, electrical signals comprising a band of frequencies, or comprising a broad spectrum or frequency sweep, may be applied, and impedance may be determined based on the multi-frequency signals, and information indicative of wound healing and/or a wound healing stage may be determined based on a multi-frequency impedance.

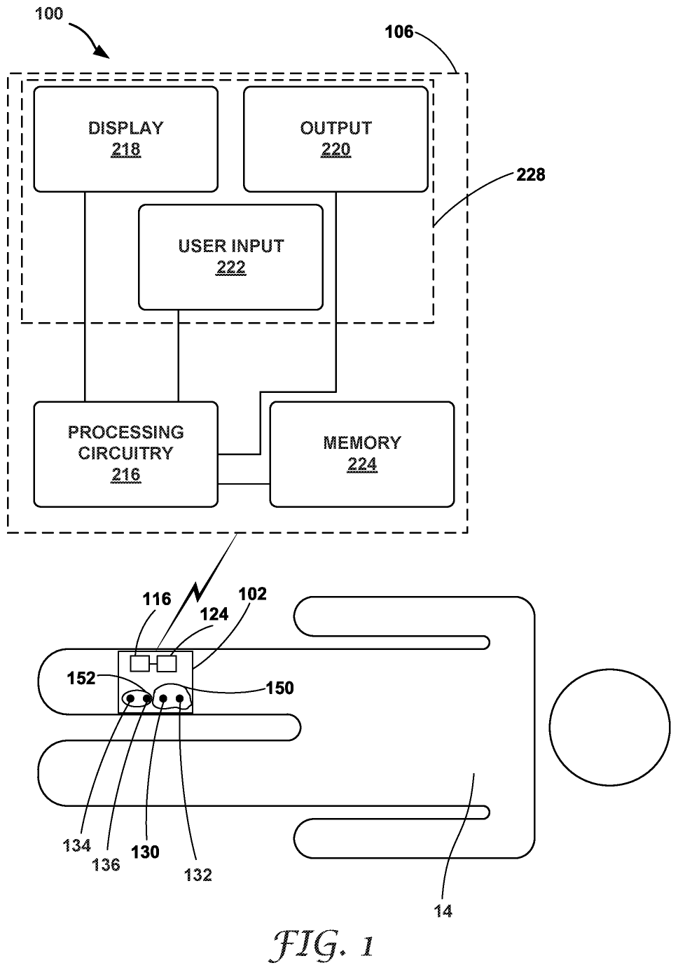

FIG. 1 is an illustration depicting an example wound monitoring system 100, in accordance with the techniques described in this disclosure. In the example shown, wound monitoring system 100 includes dressing 102 and computing device 106. Dressing 102 may be communicatively coupled, for example by a wired or a wireless connection, to computing device 106. In the illustrated example, computing device 106 may include processing circuitry 216 coupled to display 218, output 220, and user input 222 of a user interface 228.

In some examples, dressing 102 may be configured to apply electrical signals to tissue site 150 of patient 14, apply electrical signals proximate to tissue site 150, e.g., at second tissue site 152, and to detect electrical signals applied to tissue site 150 and proximate to tissue site 150. In some examples, first tissue site 150 may correspond to wounded tissue, e.g., tissue having damage to epithelial layers and/or subcutaneous tissue. In some examples, second tissue site 152 may correspond to tissue without damage, e.g., healthy tissue. In other examples, first tissue site 150 may correspond to tissue having a bruise, tissue having a rash, tissue having an infection, and the like. The electrical signals, or information corresponding to the electrical signals, may be transferred to computing device 106 for processing, for example, by a wired or wireless connection between dressing 102 and computing device 106. In some examples, dressing 102 may include processing circuitry 116 and memory 124 and may process the electrical signals without transferring the electrical signals to computing device 106.

Dressing 102 may be any type of structure that includes any of electrodes 130-136, and in some examples may comprise just electrodes 130 and 132. In some examples, dressing 102 may be a bandage including a flexible backing material, an adhesive for bonding to the skin of patient 14, and electrodes 130-136. In some examples, dressing 102 may be a foam dressing including electrodes 130-136. In other examples, dressing 102 may be a diagnostic patch, for example, a material including any of electrodes 130-136. In some examples, additional materials may be applied to patient 14 for wound monitoring, for example, sterile saline-laden gauze, a gel, or the like, placed between dressing 102 and tissue site 150. In some examples, additional materials may include treatments such as medications and/or may be at least partially electrically conductive and may enhance electrical conductivity between electrodes 130-136 and tissue site 150 and second tissue site 152.

Electrodes 130-136 may be any type of conductors capable of conducting electrical signals. For example, electrodes 130-136, alternatively referred to as electrical contacts 130-136, may be configured to apply an electrical signal to a material with which they are in contact, e.g., the skin of patient 14. Electrodes 130-136 may in addition be configured to detect, sense, capture, etc., electrical signals from a material with which they are in contact. In some examples, electrodes 130-136 may apply and detect electrical signals to/from a material in a non-contact manner, e.g., via inductance, capacitive coupling, and/or electromagnetic radiation. In some examples, electrodes 130-136 are configured to apply and detect signals in a wide frequency range. For examples, electrodes 130-136 may be configured to apply and detect electrical signals having frequencies between 1 kHz to 2 kHz, between 2 kHz to 4 kHz, between 4 kHz to 55 kHz, between kHz to 120 kHz, or having frequencies in ranges that may be greater or less than these example ranges. In some examples, electrodes 130 may be configured to apply and detect electrical signals having any frequency.

In some examples, electrodes 130 and 132 are configured to be in contact with tissue site 150 and to conduct electrical signals to and from wounded tissue, e.g., as an electrode pair. In some examples, electrodes 134 and 136 are configured to be in contact with tissue adjacent to tissue site 150, e.g., second tissue site 152, and to conduct electrical signals to and from the adjacent tissue, e.g., as an electrode pair. In some examples, electrodes 130 and 132 are different from electrodes 134 and 136, e.g., so as to be able to conduct electrical signals to and from both unwounded and wounded tissue at approximately the same time. In some examples, electrodes 130 and 132 may be the same as electrodes 134 and 136, e.g., so as to be able to conduct electrical signals to and from both unwounded and wounded tissue using the same electrodes via moving the electrodes.

Processing circuitry 216 of computing device 106, as well as processing circuitry 116 and other processing modules or circuitry described herein, may be any suitable software, firmware, hardware, or combination thereof. Processing circuitry 216 may include any one or more microprocessors, controllers, digital signal processors (DSPs), application specific integrated circuits (ASICs), field-programmable gate arrays (FPGAs), fixed function circuitry, programmable circuitry, any combination of fixed function circuitry and programmable circuitry, or equivalent discrete logic circuitry or integrated logic circuitry. The functions attributed to processors described herein, including processing circuitry 216, may be provided by processing circuitry of a hardware device, e.g., as supported by software and/or firmware.

In some examples, processing circuitry 216, as well as processing circuitry 116, is configured to determine physiological information and/or information indicative of wound healing and/or a stage of wound healing associated with patient 14. For example, processing circuitry 216 may determine an impedance, and/or impedances, of tissue site 150, based on applied electrical signals. In some examples, processing circuitry 216 may determine an impedance, and/or impedances, of a location proximate to tissue site 150 based on applied electrical signals. In some examples, processing circuitry 216 may determine one or more reactances and/or impedance phase angles of tissue site 150, a location within tissue site 150, and/or a location proximate to tissue site 150. In some examples, processing circuitry 216 may determine one or more impedance magnitudes and/or resistances of tissue site 150, a location within tissue site 150, and/or a location proximate to tissue site 150. In some examples, processing circuitry 216 may determine information indicative tissue characteristics based on the impedance, impedance magnitude, resistance, reactance, and/or impedance phase angle. In some examples, processing circuitry 216 may determine information indicative tissue characteristics such as wound healing and/or a stage of wound healing, e.g., inflammation, granulation, an amount and/or thickness of granulation tissue, re-epithelialization and/or an amount of new epithelial monolayer regenerated in tissue, an amount of size or thickness of regenerated epithelium, a relative maturation of stratus corneum, and/or an amount of corneal site de-nucleation.

Processing circuitry 216 may perform any suitable signal processing of electrical signals to filter the electrical signals, such as any suitable band-pass filtering, adaptive filtering, closed-loop filtering, any other suitable filtering or processing as described herein, and/or any combination thereof. Processing circuitry 216 may also receive input signals from additional sources (not shown). For example, processing circuitry 216 may receive an input signal containing information about treatments provided to the patient. Additional input signals may be used by processing circuitry 216 in any of the calculations or operations it performs in accordance with wound monitoring system 100. In some examples, processing circuitry 216 may be adapted to execute software, which may include an operating system and one or more applications, as part of performing the functions described herein. In some examples, processing circuitry 216 may include one or more processing circuitry for performing each or any combination of the functions described herein.

In some examples, processing circuitry 216 may be coupled to memory 224, and processing circuitry 116 may be coupled to memory 124. Memory 224, as well as memory 124, may include any volatile or non-volatile media, such as a random-access memory (RAM), read only memory (ROM), non-volatile RAM (NVRAM), electrically erasable programmable ROM (EEPROM), flash memory, and the like. Memory 224 may be a storage device or other non-transitory medium. Memory 224 may be used by processing circuitry 216 to, for example, store fiducial information or initialization information corresponding to physiological monitoring, such as wound monitoring. In some examples, processing circuitry 216 may store physiological measurements or previously received data from electrical signals in memory 224 for later retrieval. In some examples, processing circuitry 216 may store determined values, such as information indicative of epithelial tissue characteristics, or any other calculated values, in memory 224 for later retrieval.

Processing circuitry 216 may be coupled to user interface 228 including display 218, user input 222, and output 220. In some examples, display 218 may include one or more display devices (e.g., monitor, PDA, mobile phone, tablet computer, any other suitable display device, or any combination thereof). For example, display 218 may be configured to display physiological information and information indicative of epithelial tissue characteristics determined by wound monitoring system 100. In some examples, user input 222 is configured to receive input from a user, e.g., information about patient 14, such as age, weight, height, diagnosis, medications, treatments, and so forth. In some examples, display 218 may exhibit a list of values which may generally apply to patient 14, such as, for example, age ranges or medication families, which the user may select using user input 222.

User input 222 may include components for interaction with a user, such as a keypad and a display, which may be the same as display 218. In some examples, the display may be a cathode ray tube (CRT) display, a liquid crystal display (LCD) or light emitting diode (LED) display and the keypad may take the form of an alphanumeric keypad or a reduced set of keys associated with particular functions. User input 222, additionally or alternatively, include a peripheral pointing device, e.g., a mouse, via which a user may interact with the user interface. In some examples, the displays may include a touch screen display, and a user may interact with user input 222 via the touch screens of the displays. In some examples, the user may also interact with user input 222 remotely via a networked computing device.

In some examples, wound monitoring system 100 may determine information indicative of healing. For example, processing circuitry 116 and/or 216 may determine information indicative of wound healing and/or a stage of wound healing based on impedance. In some examples, determination of wound healing may be based on impedance of wounded tissue normalized by a baseline impedance of unwounded tissue. In some examples, determination of wound healing and/or a stage of wound healing may be based on a plurality of ratios of impedance of wounded tissue and one or more baseline impedances.

For example, impedance may vary because of variation in the electrical characteristics from tissue site to tissue site of an individual, e.g., different tissue locations on the same patient and/or animal, variation of tissue characteristics at different times, and variation from individual to individual, e.g., from patient to patient and/or animal to animal. For example, electrical characteristics of tissue may vary based on tissue composition and thickness, tissue water content and/or tissue hydration, ambient relative humidity at the time of measurement, and the like. In some examples, to reduce site to site and/or patient to patient impedance variation, processing circuitry 116 and/or 216 may determine a baseline impedance at or near the time of measurement of an impedance of first tissue, e.g., wounded tissue, and may normalize the first tissue impedance measurement. For example, processing circuitry 116 and/or 216 may concurrently measure a tissue baseline impedance of a second tissue, e.g., unwounded tissue, at a second tissue site which may be adjacent to the first tissue and may be used to normalize the first tissue impedance measurement.

However, measuring the baseline impedance concurrently or near concurrently with the impedance of the wounded tissue is not necessary in all examples. In some examples, a baseline impedance representative of unwounded tissue corresponding to the wounded tissue site, e.g., tissue site 150, may be measured previous to measurement of an impedance of wounded tissue at tissue site 150, and in some examples may be electronically stored for future normalization, e.g., after a wound occurs at tissue site 150. In still other examples, a baseline impedance may be derived from previously measured impedances of unwounded tissue of a representative population, e.g., an average of measured impedances of unwounded tissues at particular anatomical sites of a population of patients. In still other examples, a baseline impedance may be theoretically derived, or derived by any other appropriate techniques for representing a baseline impedance phase angle of unwounded tissue that corresponds to wounded tissue.

Generally, impedance is a complex quantity including what are referred to as “real” and “imaginary” quantities, e.g., Z=R+iX, where Z is the impedance, R is the so-called real component and is the resistance, and X is the so-called imaginary component and is the reactance. The real component of complex impedance (resistance) indicates how electrical energy is lost in a system, while the imaginary component of complex impedance (reactance) indicates how electrical energy is stored in a system. Negative reactance values indicate electrical energy storage in the form of electric fields, i.e. capacitive energy storage. The magnitude of the impedance may be calculated as the modulus of Z, e.g., the square root of ZZ*, or |Z|=\sqrt{R²+X²}, where Z* is the complex conjugate of Z. The impedance phase angle may be calculated as θ=tan⁻¹ (X/R), where θ is the impedance phase angle. An electrical component with a finite reactance X, such as unwounded tissue that may act as a capacitor, induces a phase shift θ between a voltage across the component and the current through the component. For example, an impedance phase angle for a capacitor may be a value between 0 and −90°, or equivalently between 0 and −π/2 radians (e.g., θ=−90°, or −π/2 for a pure capacitor with no resistance). In some examples, processing circuitry 116 and/or 216 may determine an impedance of wounded tissue that is resistive with substantially no reactance, e.g., such as wounded tissue in an inflammation stage. In some examples, processing circuitry 116 and/or 216 may determine an impedance of wounded tissue that is resistive with a resistance that is less than a threshold value and with substantially no reactance, e.g., such as wounded tissue in a proliferation stage. In some examples, processing circuitry 116 and/or 216 may determine an impedance of wounded tissue with a substantially negative reactance and/or substantially negative impedance phase angle, such as wounded tissue in a remodeling stage, e.g., a re-epithelialization tissue growth stage.

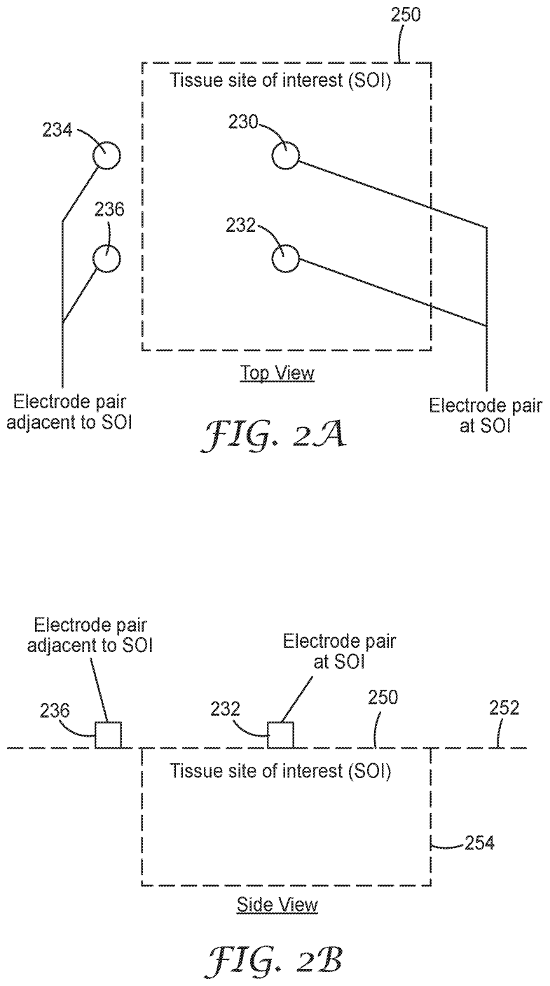

FIG. 2A is a conceptual diagram illustrating an aerial view an example tissue site 250, in accordance with one or more techniques of this disclosure. In the example shown, electrodes 230 and 232 are electrically coupled to tissue within tissue site 250, and electrodes 234 and 236 are electrically coupled to tissue adjacent to tissue site 250, e.g., tissue site 252. In some examples, an alternating electrical signal may be applied to tissue site 250 via electrodes 230 and 232, and an impedance phase angle of the wounded tissue may be determined, for example, via a circuit including electrodes 230 and 232. An alternating electrical signal may also be applied to tissue adjacent to tissue site 250 via electrodes 234 and 236, and a baseline impedance phase angle, e.g., an unwounded tissue impedance phase angle, may be determined, for example, via a circuit including electrodes 234 and 236.

FIG. 2B is a conceptual diagram illustrating a side-view of the example tissue site 250 of FIG. 2A, in accordance with one or more techniques of this disclosure. In the example shown, the electrodes 230-236 are electrically coupled to the outside of tissue surface 252. Tissue site 250 includes a tissue volume 254, e.g., wound bed, extending beneath tissue surface 254. In some examples, any or all of electrodes 236 may be attached to tissue via a dressing, and an at least partially electrically conductive material, gel, medication, and the like may be applied to any or all of electrodes 230-236, e.g., to facilitate and/or improve electrical coupling between electrodes 230-236 and the tissue. In some examples, an at least partially electrically conductive material between any or all of electrodes 230-236 may be configured to not come into electrical contact with each other, e.g., so as not to create and electrical short between two or more electrodes 230-236.

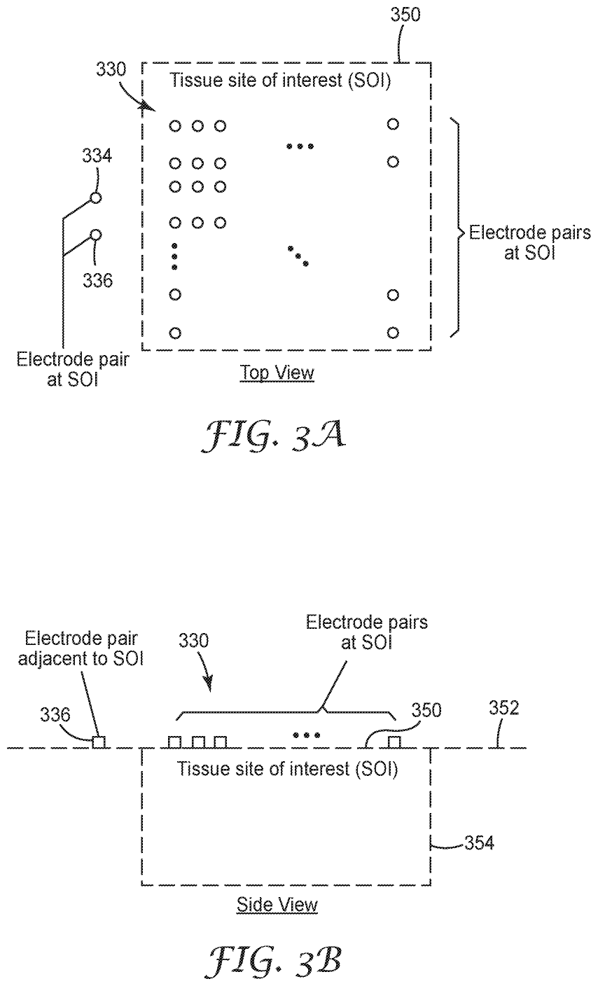

FIG. 3A is a conceptual diagram illustrating an aerial view an example tissue site 350, in accordance with one or more techniques of this disclosure. In the example shown, a plurality of electrodes 330 are electrically coupled to tissue within tissue site 350, and electrodes 334 and 336 are electrically coupled to tissue adjacent to tissue site 350, e.g., tissue site 352. In some examples, an alternating electrical signal may be applied to tissue site 350 via electrodes 330, and a plurality of impedances of wounded tissue corresponding to a plurality of locations within tissue site 350 may be determined, for example, via one or more circuits including electrodes 330. An alternating electrical signal may also be applied to tissue adjacent to tissue site 350 via electrodes 334 and 336, and a baseline impedance, e.g., an unwounded tissue impedance, may be determined, for example, via a circuit including electrodes 334 and 336.

FIG. 3B is a conceptual diagram illustrating a side-view of the example tissue site of FIG. 3A, in accordance with one or more techniques of this disclosure. In the example shown, electrodes 330, 334, and 336 are electrically coupled to the outside of tissue surface 352. Tissue site 350 includes a tissue volume 354, e.g., wound bed, extending beneath tissue surface 354. In some examples, any or all of electrodes 336 may be attached to tissue via a dressing, and an at least partially electrically conductive material, gel, medication, and the like may be applied to any or all of electrodes 330, 334, and 336, e.g., to facilitate and/or improve electrical coupling between electrodes 330, 334, and 336 and the tissue. In some examples, an at least partially electrically conductive material between any or all of electrodes 330, 334, and 336 may be configured to not come into electrical contact with each other, e.g., so as not to create and electrical short between two or more electrodes 330, 334, and 336.

In some examples, a circuit to measure and/or detect an impedance including any of electrodes 130-136, or 230-236, or 330-336 may include analog and/or digital circuit components, e.g., resistors, inductors, capacitors, filters, operational amplifiers, analog comparators, analog frequency mixers, digital phase detectors, logic components (AND gates, OR gates, NAND gates, XOR gates and the like), analog-to-digital (A/D) converters, digital-to-analog (D/A converters), microcontrollers, and the like.

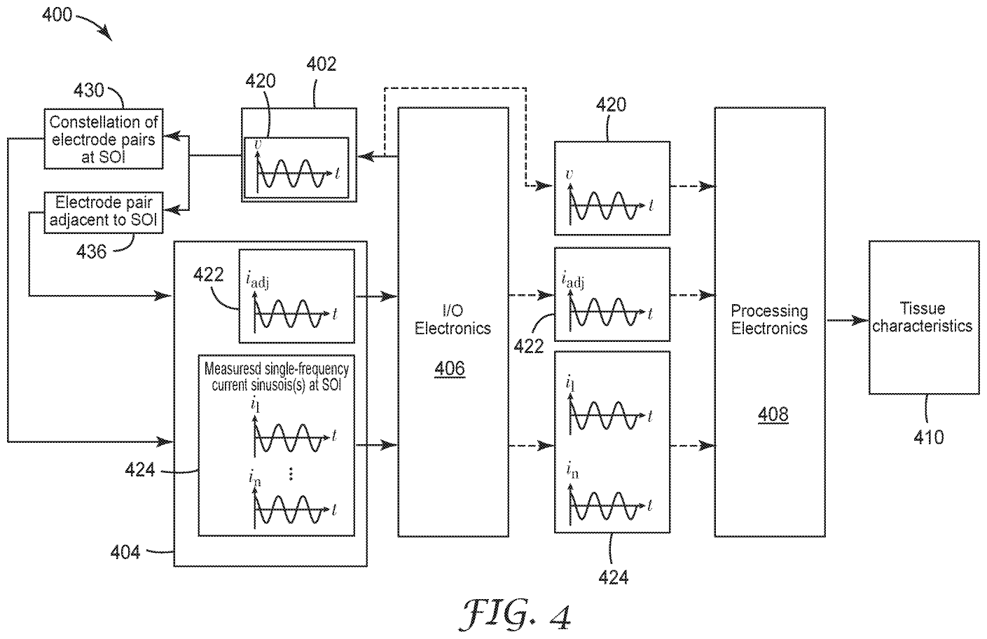

FIG. 4 is a block diagram of an example wound monitoring system 400, in accordance with the techniques described in this disclosure. In the example shown, wound monitoring system 400 includes signal generator 402, tissue site electrodes 430, second tissue site electrodes 436, signal monitor 404, input-output (I/O) electronics 406, processing circuitry 408, and output 410. Wound monitoring system may be an example of wound monitoring system 100 illustrated and described above with respect to FIG. 1.

In the example shown, signal generator 402 is configured to generate an alternating electrical signal, e.g., an electrical waveform. The electrical signal may be sinusoidal, a square wave, a pulse wave, a triangle wave, a sawtooth wave, and the like. Signal generator 402 may be configured to generate an electrical signal including one or more frequencies at any frequency, including between 1 kHz to 2 kHz, between 2 kHz to 4 kHz, between 4 kHz to 55 kHz, between 55 kHz to 120 kHz. In some examples, signal generator 402 may be configured to generate an electrical signal in a frequency range that may be greater than or less than the example ranges above. In some examples, electrical signal generator 402 may be configured to generate an electrical signal at a predetermined frequency, such as approximately 85 kHz (e.g., 85 kHz±10 kHz). In the example shown, signal generator 402 is configured to generate voltage signal 420.

In the example shown, signal generator 402 is electrically connected to tissue site electrodes 430 and second tissue site electrodes 436. Tissue site electrodes 430 may include two or more electrodes, and may be substantially similar to electrodes 130, 132, 230, 232, and 330 described above. Second tissue site electrodes 436 may include two or more electrodes, and may be substantially similar to electrodes 134, 136, 234, 236, 334, and 336 described above. Tissue site electrodes 430 and second tissue site electrodes 436 may be configured to be electrically connected to tissue, and in some examples may be attached to tissue via a dressing. Signal generator 402 may be an example of at least a portion of processing circuitry 408 described below. Signal generator 402 may be an example of at least a portion of processing circuitry 116 and/or 216 illustrated and described above, with respect to FIG. 1.

In the example shown, signal monitor 404 is electrically connected to tissue site electrodes 430 and second tissue site electrodes 436. In some examples, signal monitor 404 may be configured to determine and/or measure electrical signals, e.g., voltage signals, current signals, and the like, and signal monitor 404 may be configured to determine and/or measure electrical resistance, electrical reactance, electrical impedance, electrical impedance phase angle, and the like. For example, signal monitor 404 may be configured to measure an impedance of tissue between any two electrodes of tissue site electrodes 430, e.g., via measurement of a voltage signal and corresponding current signal of a circuit including signal generator 402, electrodes 430, and tissue to which electrodes 430 are electrically connected. In some examples, signal monitor 404 is configured to measure a plurality of impedances corresponding to a plurality of locations of tissue two which electrodes 430 are electrically connected, e.g., a tissue site.

In the example shown, signal monitor 404 is configured to measure current signal 422 via electrodes 436 and one or more current signals 424 via electrodes 430. In some examples, signal monitor 404 may derive a reactance and/or an impedance phase angle from an impedance measurement, and in other examples a reactance and/or an impedance phase angle may be derived by a different device receiving information corresponding to an impedance measurement by signal monitor 404, e.g., derived by processing circuitry 408 described below. In some examples, signal monitor 404 may measure and/or determine an impedance of unwounded tissue, e.g., a baseline impedance. For example, signal monitor 404 may measure and/or determine an impedance of tissue between two or more electrodes of second tissue site electrodes 436. Signal monitor 404 may be an example of at least a portion of processing circuitry 408 described below. Signal monitor 404 may be an example of at least a portion of processing circuitry 116 and/or 216 illustrated and described above, with respect to FIG. 1.

In some examples, signal generator 402 may be electrically connected to only tissue site electrodes 436, and signal monitor 404 may determine one or more wounded tissue impedances. A baseline impedance, e.g., of unwounded tissue, may be pre-stored, such as an expected impedance based on an average impedance of unwounded tissue from a patient population.

In some examples, signal generator 402 and signal monitor 404 may be configured to generate and measure electrical signals at a plurality of frequencies such that a plurality of impedances corresponding to the plurality of frequencies may be determined and/or measured. In other words, wound monitoring system 400 may determine wounded tissue impedance and baseline impedance as a function of frequency, e.g., via a frequency sweep.

In the example shown, I/O electronics 406 is communicatively coupled to signal generator 402, signal monitor 404, and processing circuitry 408. In some examples, I/O electronics may be included in computing device 106 illustrated and described with respect to FIG. 1 above and may be configured as an interface between electrical devices, e.g., signal generator 402, signal monitor 404, and processing circuitry 408. For example, I/O electronics 406 may be configured to communicate signal generation commands and information from processing circuitry 408 to signal generator 402, and receive information corresponding to generated electrical signals from signal generator 402 and relay the received information to processing circuitry 408. I/O electronics 406 may receive information corresponding to measurements, electrical signals and/or impedance phase angles from signal monitor 404 and relay the received information to processing circuitry 408. In the example of FIG. 4, I/O electronics 406 is configured to relay information corresponding to generated voltage signal 420 and measured current signals 422 and 424 to processing circuitry 408.

In some examples, processing circuitry 408 is configured to receive electrical signals, and/or information corresponding to electrical signals, and determine impedances based on the received information and/or signals. Processing circuitry 408 may also be configured to determine information indicative of tissue characteristics, such as wound healing and/or a stage of wound healing based on impedances. In some examples, to improve determination of information indicative of tissue characteristics, processing circuitry 408 may be configured to determine a ratio of impedances to baseline impedances. For example, processing circuitry 408 may determine information indicative of tissue characteristics based on a ratio of impedances of wounded tissue, e.g., corresponding to tissue locations to which electrodes 430 are electrically connected, to a baseline impedance, e.g., a pre-stored baseline impedance, a measured unwounded tissue impedance corresponding to tissue locations to which second tissue site electrodes 436 are electrically connected. In some examples, processing circuitry 408 may include and/or be communicatively coupled with memory, e.g., such as memory 124 and/or memory 224. In some examples, processing circuitry may be configured to determine information indicative of a stage of healing such as an inflammation healing stage, a proliferation stage, an amount of healing, an amount and/or thickness of granulation tissue, a lack of healing and/or chronicity such as a chronic wound, a remodeling stage, an amount of new epithelial coverage regenerated in tissue, and amount of size or thickness of regenerated epithelium, a relative maturation of stratus corneum, a healed stage, and the like, based on impedance.

Processing circuitry 408 may be an example of processing circuitry 216 or 116 illustrated and described above with respect to FIG. 1. In the example shown, processing circuitry is communicatively coupled to output 410. In some examples, output 410 may be substantially similar to user interface 228 of computing device 106, illustrated and described above with respect to FIG. 1. In some examples, output 410 may be configured to communicate, record, and or display values and/or information indicative of epithelial tissue characteristics, e.g., to a user, a database, network, another computing device, and the like. In some examples, any and/or all of signal generator 402, signal monitor 404, I/O electronics 406, and processing circuitry may be included in a single device.

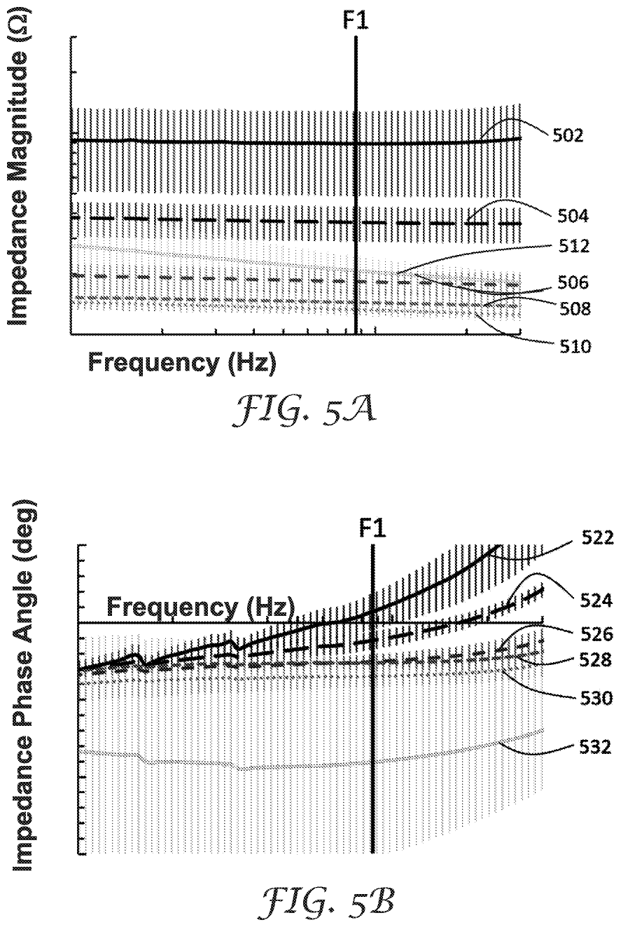

FIG. 5A is a plot of example impedance magnitudes as a function of signal frequency for a plurality of tissue sites at a plurality of times after a wound occurs at each of the tissue sites, in accordance with the techniques described in this disclosure. FIG. 5B is a plot of example impedance phase angles as a function of signal frequency for a plurality of tissue sites at a plurality of times after a wound occurs at each of the tissue sites, in accordance with the techniques described in this disclosure. The examples shown in FIGS. 5A and 5B represent the impedance amplitude and phase angle of the same impedance measurements. In the examples shown, impedance magnitude curves 502-512 and impedance phase angle curves 522-532 correspond to six different impedance measurement times after wounding at each of the eight tissue sites. Impedance amplitude curves 502-512 are the mean of wounded tissue impedance magnitude curves averaged over the eight wounded tissue sites at each respective measurement time, and impedance phase angle curves 522-532 are the mean of wounded tissue impedance phase angle curves averaged over the eight wounded tissue sites at each respective measurement time. For example, impedance magnitude curve 502 corresponds to the mean of eight impedance magnitude curves for impedances measured at “day 0,” e.g., the same day as the occurrence and/or generation of the wound. Impedance magnitude curve 504 corresponds to the mean of eight impedance magnitude curves for impedances measured at “day 3,” e.g., three days after the occurrence and/or generation of the wound. Impedance magnitude curves 506, 508, 510, and 512, corresponds to the mean of eight impedance magnitude curves for impedances measured at days 7, 10, 14, and 16, respectively. Similarly, impedance phase angle curves, 522, 524, 526, 528, 230, and 532 correspond to the mean of eight impedance phase angle curves for impedance measured at days 0, 3, 7, 10, 14, and 16, respectively. The error bars of each respective curve 502-512 and 552-532 are the standard deviation of the eight averaged impedance magnitude and phase angle curves, respectively.

The examples shown in FIGS. 5A and 5B illustrate that the impedance measured at different times may be used to determine information indicative of a stage of wound healing, e.g., the inflammation, proliferation, remodeling, and healed stages may be distinguishable based on the impedance of the wound. For example, as a wound progresses from the inflammation stage just after wounding to the proliferation stage, the impedance magnitude reduces, as exemplified by the progressive impedance magnitude reduction of curves 502-508 from day 0 to day 12. During the inflammatory and proliferation stages, the impedance is substantially resistive with little reactance, as exemplified by the impedance phase angle of curves 522-528 being relatively close to zero degrees. In the examples shown, as the wound re-epithelializes, as the tightly joined epithelial layers are gradually restored and provide an electrical barrier that facilitates electrical, e.g., capacitive charging. This epithelial stage is exemplified by the increasingly negative phase angles curves 530 and 532 as well as an increase in impedance magnitude between curves 510 to 512.

In the examples shown in FIGS. 5A and 5B, the impedances are measured over a range of frequencies. In some examples, the impedance measurements curves are statistically differentiable from each other over a frequency range from 10 kHz to 200 kHz. In the examples shown in FIGS. and 5B, an impedance measurement at a predetermined frequency within the range from 10 kHz to 200 kHz, e.g., impedance amplitude and phase angle, may be evaluated and/or used to determine information indicative of a stage of healing. In the examples shown, information indicative of a stage of healing may be determined from impedance magnitudes and phase angles at frequency F1, where F1 may be any frequency between 10 kHz and 200 kHz. In some examples, F1 may be 85 kHz.

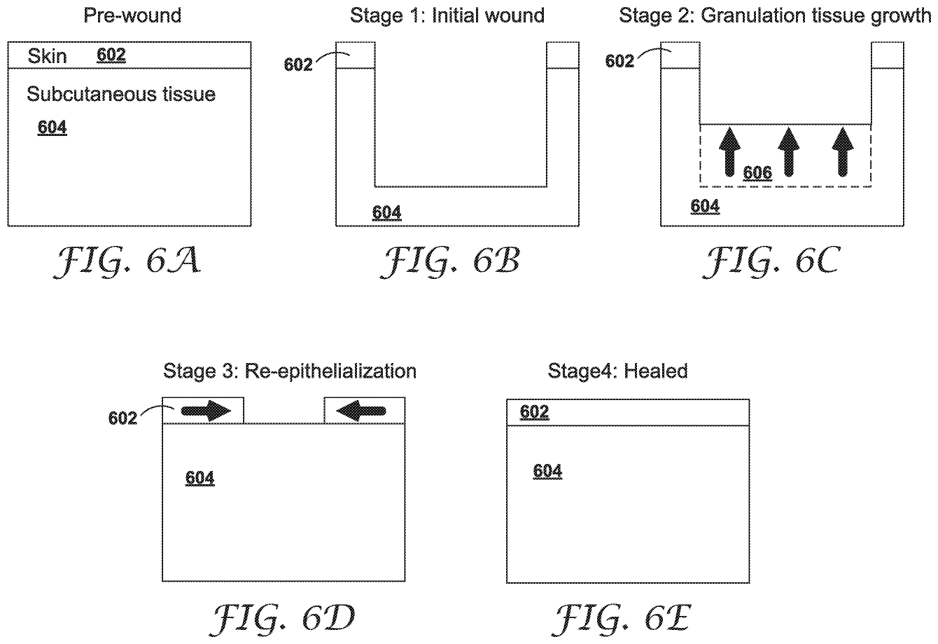

FIGS. 6A-6E are conceptual cross-sectional schematic diagrams of a wound site at various stages of healing, in accordance with the techniques described in this disclosure. In some examples, a wound may heal in a progression from FIG. 6B to 6E, representing stages of healing. FIG. 6A is a conceptual cross-sectional schematic diagram of skin 602 and subcutaneous tissue 604 before wounding. FIG. 6B is a conceptual cross-sectional schematic diagram of skin 602 and subcutaneous tissue 604 of wounded tissue close to the time of wounding and illustrating removal of a portion of skin 602 and subcutaneous tissue 604. The example shown in FIG. 6B may correspond to a first stage of healing, or an inflammation stage, in which damages or removed tissue may exhibit hemostasis and an inflammatory response. During this first stage, the electrical impedance of the tissue may be substantially resistive with substantially little reactance, e.g., due to the removal of skin 602. As the wound heals, it may grow granulation tissue during a second, or proliferation stage, as illustrated via the filling in of the wound's void space with tissue 606 in FIG. 6C. The electrical impedance of subcutaneous tissue 604 may still be substantially resistive with substantially little reactance, but the resistance may be reduced relative to the first stage, e.g., granulation tissue 606 may be more conductive than the native tissue. The electrical impedance may still have substantially little reactance because granulation tissue 606 may be disorganized and lack structures that facilitate electrical charging.

The example shown in FIG. 6D illustrates a third, or remodeling stage (i.e., re-epithelialization) in which skin 602, e.g., tightly joined epithelial layers, regenerate atop the newly formed granular tissue. For example, the epithelial layers may be gradually restored by development of an epithelial monolayer starting at the wound margin and then a thickening of this monolayer through epithelial proliferation, and then with a de-nucleation of cells contained in the epithelium's topmost layer. The growing epithelial layer may provide an electrical boundary that may facilitate electrical charging, and the electrical impedance during remodeling may exhibit an increasing negative reactance (e.g., capacitance characterized by the phase of an electrical voltage and/or current passing through the tissue lagging the input electrical signal voltage and/or current) as the epithelial layers are gradually restored. The example shown in FIG. 6E illustrates an example fourth, or healed stage, in which the electrical impedance of the substantially re-epithelialized and healed wound may be restored to the original impedance of the tissue site before the wound and/or an impedance substantially similar to nearby unwounded tissue.

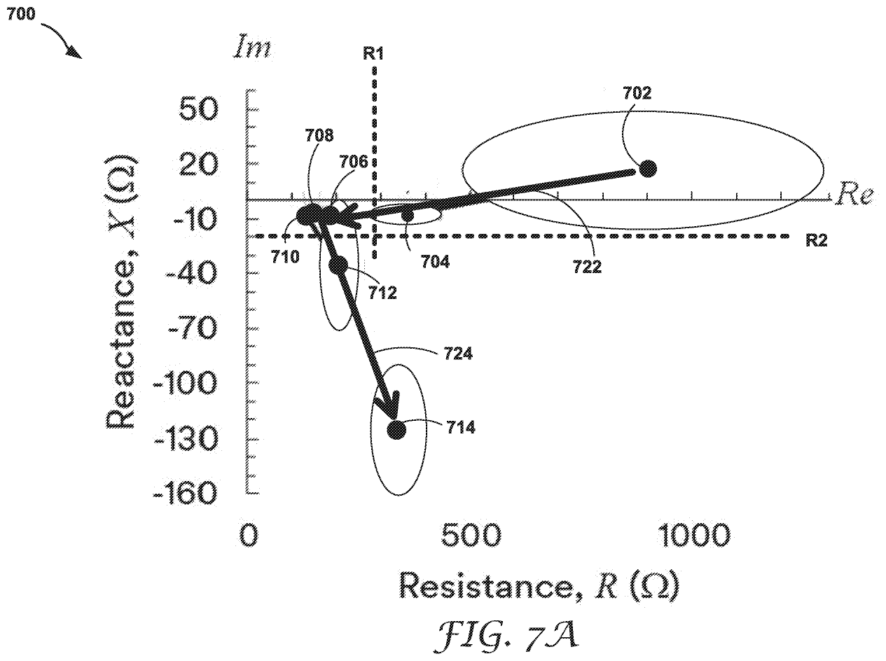

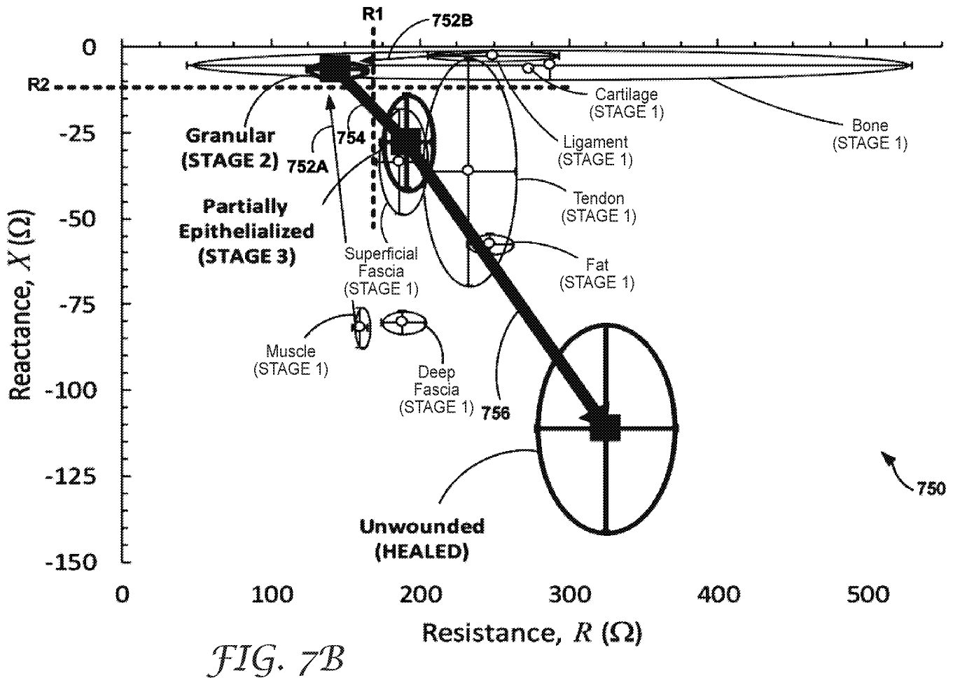

FIG. 7A is a plot 700 of example complex impedances of a tissue site at a predetermined frequency F1 measured at a plurality of times after a wound at the tissue site, in accordance with the techniques described in this disclosure. The example shown in FIG. 7 illustrates a progression of the impedance of a tissue site at a predetermined frequency F1 as a wound progresses through multiple stages of healing. In the example shown, the impedance measurements 702-712 correspond to the average value of the impedance measurements at the eight wounded tissue sites at frequency F1 and corresponding to the six times after the occurrence of the wound, e.g., days 0, 3, 7, 10, 14, and 16, illustrated and described above with reference to FIGS. 5A-5B. Plot 700 is a resistance vs. reactance plot in which each mean impedance measurement value 702-712, e.g., averaged over the eight wounded tissue sites, is represented by a resistance (i.e., the real component of its complex impedance) and reactance (imaginary component of its complex impedance) value pair. In other words, plot 700 is a two-dimensional (2D) plot of the mean impedance measurements at the different times, and the dimensions of plot 700 are resistance and reactance. In the examples shown, the mean impedance measurements 702-712 are the values at the frequency F1, e.g., 85 kHz.

In the example shown, the progression of the impedance vs. time on plot 700 follows “paths” 722 and 724, e.g., changes in the resistance and reactance values analogous to a vectors in the 2D resistance-reactance plane. Paths 722 and 724 may correspond to changes in impedance of a wounded tissue site as the wounded tissue progresses through different stages of healing. In the example shown, a wounded tissue site may begin, e.g., around the time of the occurrence of the wound, by exhibiting no tissue growth in stage 1 (inflammatory), and the electrical characteristics of the wounded tissue may be primarily resistive with little reactance. The resistance/reactance values of impedance measurements 702 and 704 at day 0 and day 3 from the occurrence of the wound, respectively, may comprise information indicative of the wounded tissue site being in the first stage, e.g., the inflammatory stage of healing.

As the wound site heals and progresses to stage 2 (proliferation, e.g., granulation), the impedance may “follow” path 722 in which the reactance remains close to zero and the resistance reduces. In other words, the wounded tissue site may change from substantially resistive to an increased conductivity during stage 1. In some examples, a transition from stage 1 to stage 2 may be determined based on a change in resistance of the wounded tissue site to less than a threshold value R1. In the example shown, the resistance value of impedance measurement 706, corresponding to day 7, is less than threshold resistance R1 and has a reactance close to zero, indicating that the wounded tissue site has progressed from stage 1 to stage 2. In some examples, the impedance of the wounded tissue site may remain relatively conductive during stage 2. In the example shown, the values of impedance measurements 706, 708 and 710, corresponding to day 7, day 10 and day 14, respectively, have relatively low resistance values and reactance values close to zero, e.g., relatively close to the origin in plot 700. The resistance/reactance values of impedance measurements 706, 708 and 710 may comprise information indicative of the wounded tissue site being in the second stage, e.g., the proliferation stage of healing.

As the wounded tissue site progresses in healing to stage 3, e.g., remodeling/re-epithelialization, the impedance of the wounded tissue site may “turn” away from the origin to increasingly negative reactance values. For example, impedance measurements of the wounded tissue site may follow path 726, which changes in direction to be substantially negative along the reactance axis. In the example shown, impedance measurement 712 includes a relatively low resistance value and an increasingly negative reactance value. The resistance/reactance values of impedance measurement 712, corresponding to day 16, may comprise information indicative of the wounded tissue site being in the third stage, e.g., the remodeling stage of healing. During the third stage, the impedance may remain on path 724. For example, as more epithelium grows over the wounded tissue site, the wounded tissue site may transition from being substantially conductive to being substantially capacitive (exhibiting negative reactance). In some examples, when the wounded tissue site has fully re-epithelialized, the tissue site's electrical characteristics, e.g., impedance will be similar to that of pre-wounded and/or nearby, unwounded tissue, illustrated on plot 700 as impedance 714 in the fourth, fully healed stage.

FIG. 7B is a plot 750 of example complex impedances of a plurality of tissue types at a predetermined frequency F1 measured at a plurality of times after a wound occurs at the tissue site, in accordance with the techniques described in this disclosure. The example shown in FIG. 7B illustrates a progression of the impedance of a tissue site at a predetermined frequency F1 as a wound progresses through multiple stages of healing and may be substantially similar to plot 700 of FIG. 7A, the difference being that the resistance and reactance values for stage 1 may vary significantly depending on tissue type. The path from stage 1 to stage 2 may be different based on the tissue “exposed,” where the electrical signal is applied to the exposed tissue. Similar to plot 700, plot 750 is a two-dimensional (2D) plot of impedance measurements at the different times. In the example shown in FIG. 7B, as a tissue site progresses through stages of healing, the progression of the impedance vs. time on plot 750 may follows paths 752-756 based on the tissue type of the wound site. In the example shown, a wounded tissue site may begin, e.g., around the time of the occurrence of the wound, by exhibiting no tissue growth in stage 1 (inflammatory), and the electrical characteristics of the wounded tissue may vary based on the type of tissue being measured. For example, the resistance/reactance values of muscle tissue in stage 1 may follow path 752A as the tissue site heals and progresses from stage 1 to stage 2, namely, proliferation. By contrast, the resistance/reactance values of ligament tissue in stage 1 may be different from those of muscle tissue and may follow path 752B as the tissue site heals and progresses from stage 1 to stage 2.

As the wound site heals and progresses to stage 2 (proliferation, e.g., granulation), the wounded tissue site may increase in conductivity (e.g., via reduced resistivity) during stage 1 and reduce in capacitance (e.g., via reduced reactance). In some examples, a transition from stage 1 to stage 2 may be determined based on a change in resistance of the wounded tissue site to less than a threshold value R1 and a change in reactance of the wounded tissue site to greater than a threshold value R2, illustrated as granular stage 2 in FIG. 7B. Resistance/reactance values of impedance measurements of the wounded tissue site being less than the threshold value R1 and greater than the threshold value R2, respectively, may comprise information indicative of the wounded tissue site being in the second stage, e.g., the proliferation stage of healing.

As the wounded tissue site progresses in healing to stage 3, e.g., remodeling/re-epithelialization, the impedance of the wounded tissue site may “turn” away from the origin to increasingly negative reactance values, similar to plot 700 described above. For example, impedance measurements of the wounded tissue site may follow path 754, which changes in direction to be substantially negative along the reactance axis. During the third stage, the impedance may follow path 756. For example, as more epithelium grows over the wounded tissue site, the wounded tissue site may be both increasingly conductive and increasingly capacitive (negative reactance). In some examples, when the wounded tissue site has fully re-epithelialized, the tissue site's electrical characteristics, e.g., impedance will be similar to that of pre-wounded and/or nearby, unwounded tissue, illustrated on plot 750 as unwounded (or healed) impedance in the fourth, fully healed stage.

In some examples, a single impedance value of the wounded tissue at a point in time may not be enough to distinguish between stages of healing. For example, the impedance values of superficial fascia tissue or fat tissue may be similar for a wound site in stage 1 or stage 3, e.g., the impedance values of superficial fascia tissue or fat tissue may lie along path 754 and/or path 756. In some examples, information indicative of a wound site being in the first stage, e.g., the inflammatory stage, may comprise resistance/reactance values measured to be greater than threshold value R1 and less than threshold value R2, respectively, and a determination that the wound site has not yet reached stage 2, e.g., proliferation. For example, wound monitoring system 400 may track impedance measurements and stages of healing of the wound site over time, and may determine that the wound is in, or remains in, the first stage based on the lack of a previous stage 2 determination and the resistance/reactance values of the impedance measurement to be greater than threshold value R1 and less than threshold value R2, respectively. Similarly, information indicative of a wound site being in the third stage, e.g., the remodeling stage, may comprise resistance/reactance values measured to be greater than threshold value R1 and less than threshold value R2, respectively, and a determination that the wound site has already progressed through stage 2, e.g., proliferation. For example, wound monitoring system 400 may track impedance measurements and stages of healing of the wound site over time, and may determine that the wound has progressed through stage 2, e.g., via previous impedance measurements in which the resistance/reactance values of the impedance measurement were less than threshold value R1 and greater than threshold value R2, respectively. Then, if the resistance/reactance values of the current, e.g., most recent, impedance measurement greater than threshold value R1 and less than threshold value R2, respectively, wound monitoring system 400 may determine that the wound site is in stage 3.

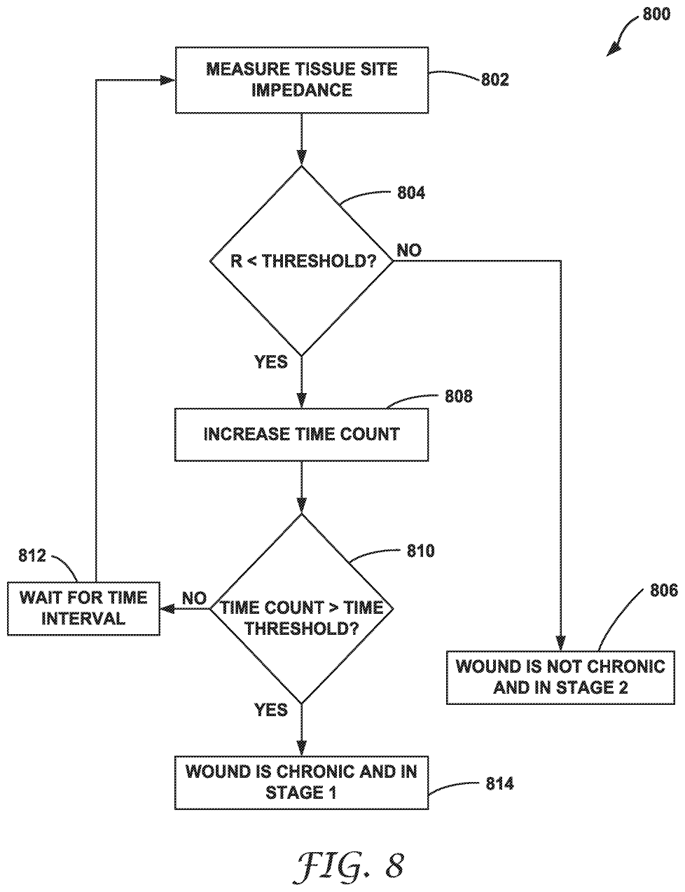

FIG. 8 is a flowchart of an example method 800 of monitoring tissue characteristics, in accordance with one or more techniques of this disclosure. The example method is described with reference to FIGS. 1-4. The example method is described below as being performed by processing circuitry 408 executing the steps of the method. In some examples, the example method may be performed, by a computing device, such as computing device 106, executing the steps of the method, or by a user and/or clinician executing the steps of the method.

Processing circuitry 408 may cause signal monitor 404 to measure an impedance at a tissue site (802). For example, user, a patient, and/or a clinician may couple electrodes 130 and 132 to tissue site 150, e.g., wounded tissue. In some examples, a user, a patient, and/or a clinician may couple electrodes 134 and 136 to tissue adjacent to tissue site 150, e.g., second tissue site 152. In some examples, electrodes 130 and 132 may be included in a dressing and attached to would site of interest 150 via the dressing. In some examples, a user may apply a material to electrodes 130-136 and/or tissue site 150 or tissue adjacent to tissue site 150 to improve electrical coupling between electrodes 130, 132, 134, and 136 and the tissue. In some examples, a user may couple a plurality of electrodes 330 to a tissue site 350 allowing for a plurality of applied electrical signals corresponding to a plurality of locations within tissue site 350 and measurement and determination of a plurality of impedance phase angles at the plurality of locations within tissue site 350.

Processing circuitry 408 may cause signal generator 402 to apply an electrical signal to tissue site 150 via electrodes 130 and 132. In some examples, signal generator 402 may apply an electrical signal, e.g., a baseline electrical signal, to tissue adjacent to tissue site 150 via electrodes 134 and 136. In some examples, signal generator 402 may apply the baseline electrical signal at substantially the same time as the electrical signal applied to tissue site 150, and in other examples signal generator 402 may apply the baseline electrical signal at a different time than the electrical signal applied to tissue site 150. In some examples, memory 124 and/or 224 and may store the baseline electrical signal, e.g., via memory 224 and/or 124. In some examples, signal generator 402 may apply a sinusoidal waveform electrical signal having a single frequency, e.g., substantially at or near 85 kHz, via electrodes 130-136. In other examples, signal generator 402 may apply a waveform that may differ from a sinusoidal waveform, e.g., a square wave, sawtooth wave, triangle wave, and the like, and a waveform that may include a plurality of frequencies.

In some examples, a plurality of electrical signals may be applied via electrodes 130-136, 330, and/or 334-336 at a plurality of predetermined times, e.g., allowing for measurement and/or determination of a plurality of impedance phase angles at the plurality of predetermined times so as to allow for tracking and/or monitoring of healing of a wound site.

Processing circuitry 408 may cause signal monitor 404 to measure the electrical signal applied to tissue site 150 via electrodes 130 and 132. In some examples, a voltage signal is applied via electrodes 130-136, and signal monitor 404 measures a resulting current signal, e.g., current signals 422 and 424. An impedance may be determined based on the applied electrical signal, e.g., via measurement of the resulting signal. For example, an impedance magnitude may be determined based on the amplitudes of the applied voltage signal and measured current signal, and an impedance phase angle may be determined based on a phase delay between the applied voltage signal and the measured current signals. Resistance (R) and reactance (X) values can be computed based on these measured impedance magnitude (|Z|) and phase angle (θ) values using the relationships R=|Z| cos θ and X=|Z| sin θ. In other examples, an impedance magnitude may be determined based on any suitable technique. In some examples, processing circuitry may cause the measured impedance to be stored, e.g., in memory included with, or communicatively coupled to processing circuitry 408. In some examples, processing circuitry 408 may cause the impedance measurement of wound site 150 to be stored along with other information, such as a time and date at which the impedance was measured, an indicator of wound site 150 such as a name and/or location, information relating to the equipment and/or methods and/or settings used with which to make the impedance measurement, and any other suitable information.

Processing circuitry 408 may determine whether the measured resistance is less than a threshold resistance (804). For example, processing circuitry 408 may determine that the resistance of tissue site 150 is less than 250 Ohms (Ω) or some other threshold value established based on species wound location, tissue type, and/or patient demographic profile. Processing circuitry 408 may determine that wounded tissue at tissue site 150 is not chronic based on determining that the measured resistance is less than the resistance threshold (806). As described above, the resistance of tissue site 150 being less than the threshold indicates that granulation tissue is forming, and the wound has progressed from stage 1 (inflammation) to stage 2 (proliferation). For example, tissue site 150 has progressed along path 722 from having a resistance greater than threshold resistance R1 indicative of stage 1 to having a resistance less than threshold resistance R1 indicative of stage 2, as illustrated and described with reference to FIG. 7. In some examples, the threshold resistance may be based on the resistance of second tissue site 152, e.g., adjacent unwounded, healthy tissue. For example, processing circuitry 408 may cause signal generator 402 to apply a voltage signal to electrodes 134 and 136 and may cause signal monitor 404 to measure an impedance of second tissue site 152, e.g., a baseline and/or comparative impedance measurement of unwounded, healthy tissue.

In some examples, processing circuitry 408 may determine that the resistance of tissue site 150 is not less than the threshold resistance and may increase a time count (808). In some examples, the time count may be stored, e.g., as a value in memory included with, or communicatively coupled to processing circuitry 408. For example, for a first measurement of impedance at tissue site 150, processing circuitry 408 may determine that the time count is zero and may increase a time count by a time interval based on determining that the resistance of the measurement impedance is not less than the threshold resistance. Processing circuitry may then cause signal generator 402 and signal monitor 404 to apply and measure electrical signals to measure the impedance of tissue site 150 a second time after waiting for the time interval (812). In some examples, the time interval may be predetermined, or the time interval may be based on user input, or processing circuitry 408 may determine a variable time interval, e.g., based on tissue site 150 impedance history and/or other information such as user input, or the time interval may be based on any other suitable method. In some examples, the time interval may be daily. For example, processing circuitry 408 may cause the impedance to be measured, such as described above, once per day. If the first measurement, e.g., day 0, is not less than the resistance threshold, processing circuitry 408 may cause the impedance to be measured a second time at day 1, one day later, e.g., a second impedance measurement. If the resistance of the second impedance measurement at day 1 is not less than the resistance threshold, processing circuitry 408 may cause the impedance to be measured a third time at day 2, one day later, and so on.

Processing circuitry 408 may determine whether the time count is greater than a time threshold (810). In some examples, the time threshold may be predetermined, or the time threshold may be based on user input, or processing circuitry 408 may determine a time threshold, e.g., based on tissue site 150 impedance history and/or other information such as user input, or the time threshold may be based on any other suitable method. If processing circuitry 408 determines that the time count is not greater than the time threshold processing circuitry 408 may cause signal generator 402 and signal monitor 404 to apply and measure electrical signals to measure the impedance of tissue site 150 after waiting for the time interval (812), and method 800 then proceeds to follow the flow described above at 802. If processing circuitry 408 determines that the time count is greater than the time threshold processing circuitry 408 may determine that tissue site 150 is in a prolonged inflammatory healing stage, which may be an early indicator that tissue site 150 may include a chronic wound, e.g., the wounded tissue is chronic, not healing, granulation tissue is not forming, or some other healing problem or issue has occurred and/or is occurring.

In some examples, processing circuitry 408 may perform the steps of method 800 and determine information indicative of the stage of healing of tissue site 150 based on multiple impedance measurements, e.g., a first impedance measurement at a first time and a second impedance measurement at a second time different from the first time by a time interval. Processing circuitry may determine that the stage of healing is chronic wound stage, e.g., that the wound is chronic and is not healing, based on the resistance and/or impedance magnitude of a plurality of impedance measurements being greater than a threshold resistance and/or threshold impedance magnitude, the reactance of a plurality of impedance measurements being less than a reactance threshold, over an amount of time or after a predetermined number of time intervals have occurred. In some examples, processing circuitry 408 may output an indication of the stage of healing of tissue site 150, e.g., via display 218, a user interface, or via any suitable method of indicating the determined stage of healing of tissue site 150.

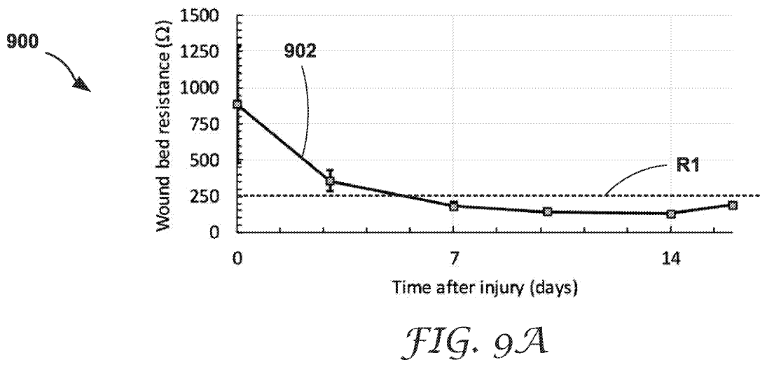

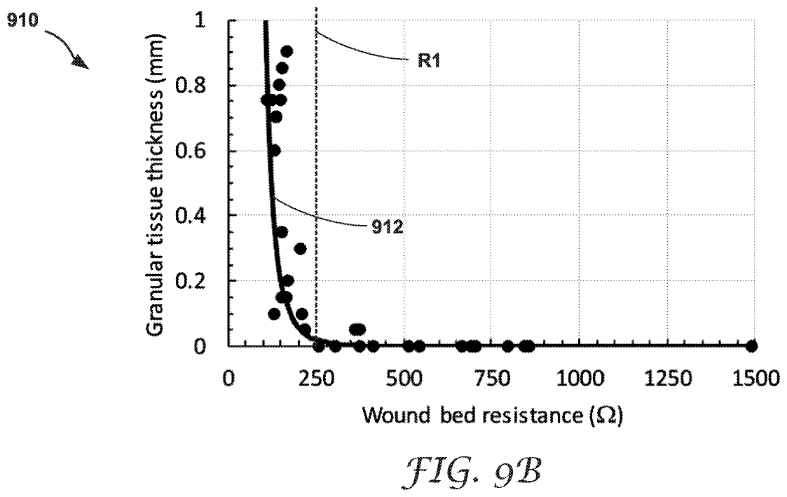

FIG. 9A is a plot 900 of example wound bed resistances at a predetermined frequency as a function of time for a tissue site, in accordance with the techniques described in this disclosure. Plot 900 includes resistance curve 902, e.g., a measured wound bed resistance as a function of time. FIG. 9B is a plot 910 of example granulation tissue thicknesses as a function of measured wound bed resistance for a tissue site at a plurality of times, in accordance with the techniques described in this disclosure. Plot 910 includes granulation thickness versus resistance curve 912, e.g., a plot of granulation thickness measured at or near the time of impedance measurements. The examples of FIGS. 9A and 9B illustrate a correlation between the resistance of the wound tissue and the thickness of the granulation tissue that has developed in the wound bed. For example, curve 912 illustrates a significant increase, e.g., bend in the curve, above resistance value R1. In some examples, R1 may be 250Ω. In some examples, R1 may be a threshold resistance that may be used to predict whether granulation tissue growth is occurring or not, e.g., per method 800 described above. In some examples, identification of the threshold resistance R1 allows for real-time determination of transitions from wound healing stage 1 (inflammation) to wound healing stage 2 (proliferation).

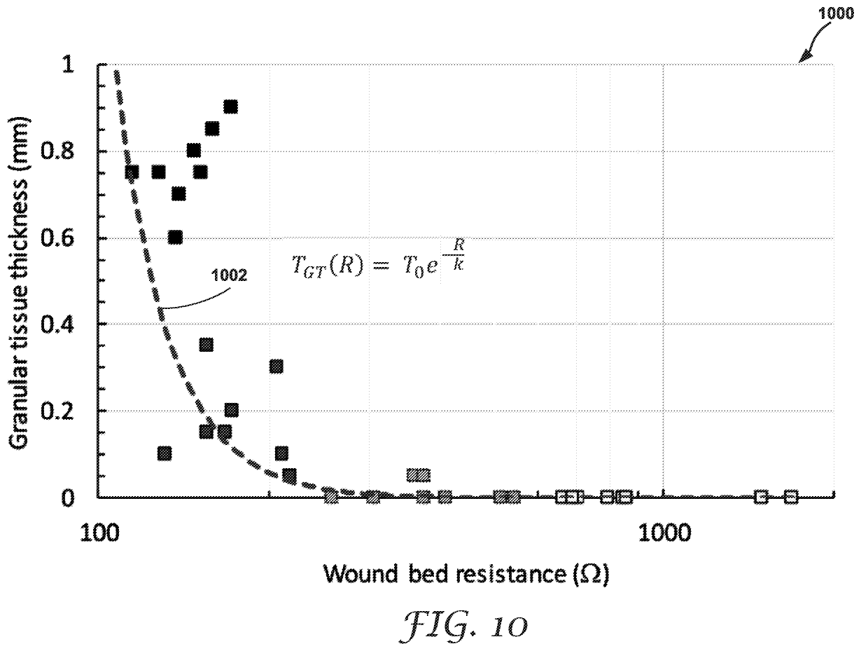

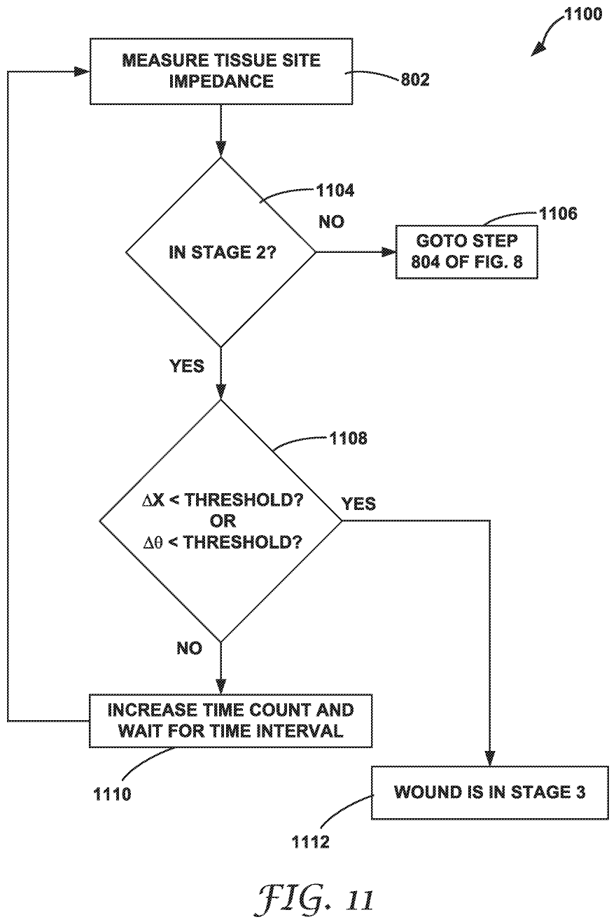

FIG. 10 is a plot 1000 of example granulation tissue thickness as a function of wound bed resistance measured at a predetermined frequency for a tissue site 150 at a plurality of times overlaid with an exponential curve fit 1002 of the granulation tissue thicknesses (T_{GT}) as a function of measured wound bed resistance (R) given a maximum possible thickness (T₀), e.g., the original wound depth, and a growth constant (k), in accordance with the techniques described in this disclosure. In some examples, the resistance may indicate an amount and/or a thickness of granulation tissue, and curve 1002 may be used to predict and/or determine the amount and/or thickness of granulation tissue. In other words, the resistance of the granulation tissue may decrease exponentially as a function of the amount and/or thickness of the granulation tissue. In some examples, processing circuitry 408 may be configured to determine the amount and/or thickness of the granulation tissue based on the resistance of tissue site 150.

FIG. 11 is a flowchart of an example method 1100 of monitoring tissue characteristics, in accordance with one or more techniques of this disclosure. The example method is described with reference to FIGS. 1-4 and 8. The example method is described below as being performed by processing circuitry 408 executing the steps of the method. In some examples, the example method may be performed, by a computing device, such as computing device 106, executing the steps of the method, or by a user and/or clinician executing the steps of the method. In some examples, method 1100 may be performed to determine whether wounded tissue, e.g., tissue site 150 is in wound healing stage 3 (remodeling/re-epithelialization).

Processing circuitry 408 may cause signal monitor 404 to measure an impedance at a tissue site 150 (802), e.g., similar to method 800 described above. Processing circuitry 408 may determine whether tissue site 150 is in stage 2 (proliferation) of wound healing (1104). For example, methods 800 and 1100 may be performed simultaneously or concurrently, and processing circuitry 408 may determine whether tissue site 150 is in stage 2 based on a determination that tissue site 150 is not chronic at 806. In some examples, processing circuitry 408 may determine whether tissue site 150 is in stage 2 based on the resistance being less that the threshold resistance, such as described above with respect to method step 804, and before method step 806. In some examples, processing circuitry 408 may determine whether tissue site 150 is in stage 2 based on previously storing the stage of healing of tissue site 150, e.g., in memory, at step 806 of method 800. Processing circuitry 408 may then determine that tissue site 150 is not in stage 2, and may proceed with method 800 at step 804, e.g., if tissue site 150 is in stage 1.

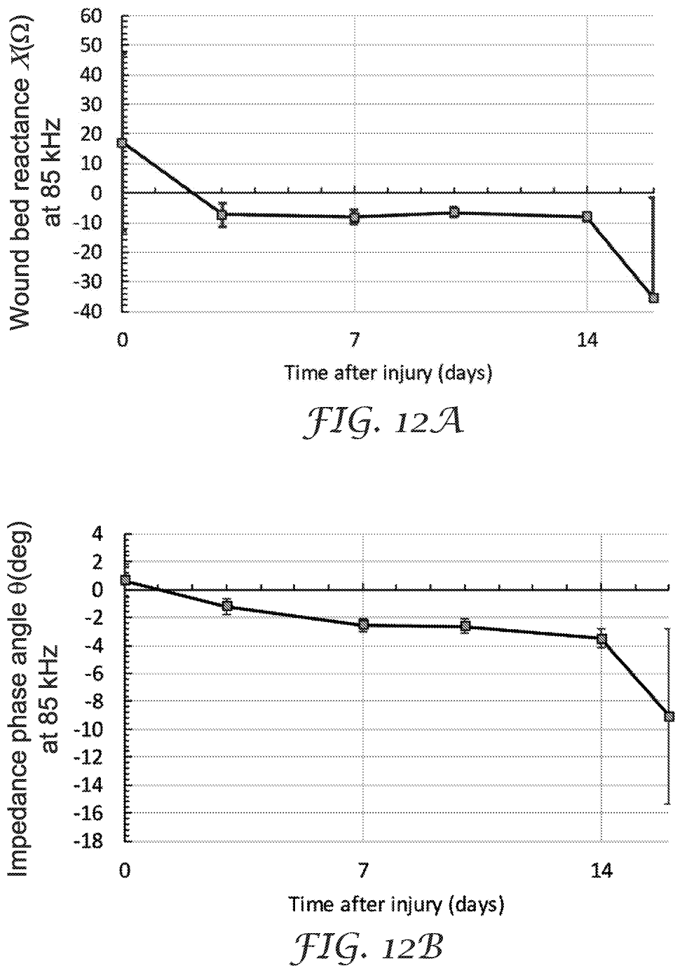

Processing circuitry 408 may then determine that tissue site 150 is not in stage 2 (1104), and may determine whether a rate of change of the reactance (ΔX) of tissue site 150 is less than a threshold reactance rate of change (e.g., a more highly negative rate of change of the reactance) and/or a rate of change of impedance phase angle (Δθ) of tissue site 150 is less than a threshold impedance phase angle rate of change (e.g., a more highly negative rate of change of the phase angle) (1108). Regarding the reactance rate of change, tissue typically exhibits capacitive behavior, e.g., the reactance is negative. As such, processing circuitry 408 may determine whether the reactance is less than, e.g., more negative than a negative reactance rate of change such that the reactance is changing to be more negative at an increasingly negative rate or is more negatively sloped such as illustrated between stages 2 and 3 in FIG. 7 or after day 14 in FIG. 12A. In some examples, processing circuitry 408 may retrieve a history of impedance measurements from memory, such as those taken at 802 during performance of methods 800 and/or 1100 and determine a rate of change as a function of time based on the current impedance measurement from 802 and one or more previous impedance measurement.

Processing circuitry 408 may determine that the rate of change of the reactance of tissue site 150 is not less than a threshold reactance rate of change and/or the rate of change of impedance phase angle of tissue site 150 is not less than a threshold impedance phase angle rate of change, and may increase a time count and cause signal monitor 404 to measure an impedance at a tissue site 150, e.g., per method step 802, after waiting for a time interval (1110). Method 900 may then repeat at method step 802.

Processing circuitry 408 may determine that the rate of change of the reactance of tissue site 150 is less than a threshold reactance rate of change or that the rate of change of impedance phase angle of tissue site 150 is less than a threshold impedance phase angle rate of change, and may determine that tissue site 150 is in stage 3 of wound healing (1112). In other words, processing circuitry 408 may determine a remodeling/re-epithelialization tissue growth stage based on the rate of change of the reactance being less than the threshold reactance rate of change, or the impedance phase angle being less than the threshold impedance phase angle rate of change, between first and second impedance measurements.

FIG. 12A is a plot of example reactances measured at a predetermined frequency as a function of time for a tissue site, in accordance with the techniques described in this disclosure. FIG. 12B is a plot of example impedance phase angles measured at a predetermined frequency as a function of time for a tissue site, in accordance with the techniques described in this disclosure. The examples of FIGS. 12A and 12B illustrate an increased negative rate of change of reactance and impedance phase angle, respectively, of a tissue site transitioning to stage 3 (remodeling/re-epithelialization). In the examples shown, the rate of change of the reactance and impedance phase angle of the measured impedance of tissue site 150 increases negatively at to less than −4 Ω/day and −0.7°/day, respectively.

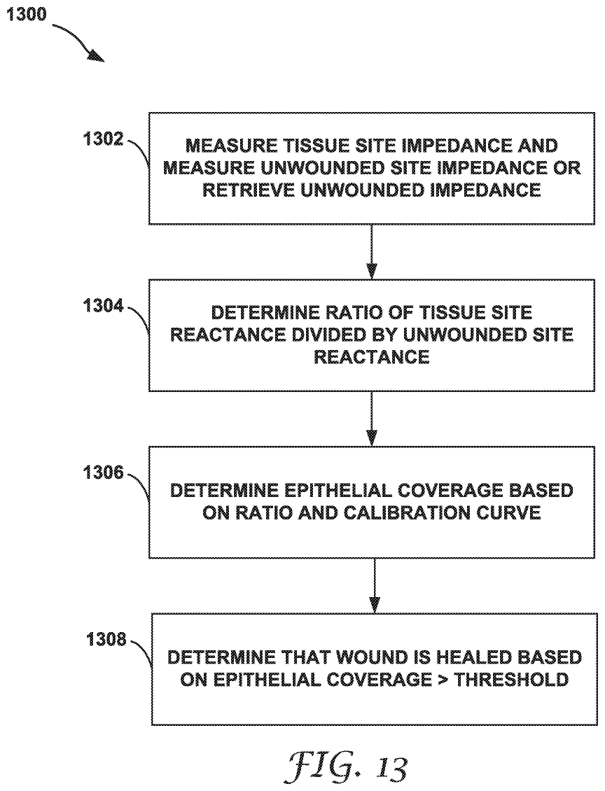

FIG. 13 is a flowchart of an example method 1300 of monitoring tissue characteristics, in accordance with one or more techniques of this disclosure. The example method is described with reference to FIGS. 1-4, 14A, and 14B. The example method is described below as being performed by processing circuitry 408 executing the steps of the method. In some examples, the example method may be performed, by a computing device, such as computing device 106, executing the steps of the method, or by a user and/or clinician executing the steps of the method. In some examples, method 1300 may be performed to determine epithelial coverage and whether wounded tissue, e.g., tissue site 150 is in wound healing stage 4 (healed).

Processing circuitry 408 may cause signal monitor 404 to measure an impedance at a tissue site 150 and a baseline impedance at a tissue site 152, e.g., similar to method step 802 described above (1302). Processing circuitry 408 may determine a ratio of the reactance value of tissue site 150 to the reactance value of the baseline impedance of tissue site 152, and/or processing circuitry 408 may determine a ratio of the impedance phase angle value of tissue site 150 to the impedance phase angle value of the baseline impedance of tissue site 152 (1304).

Processing circuitry 408 may determine epithelial coverage of tissue site 150 based on a comparison of the determined reactance ratio, or impedance phase angle ratio, and an epithelial coverage calibration curve and/or data set (1306). For example, processing circuitry 408 may record impedance measurements over time, e.g., per methods 800, 900, and/or 1100. In some examples, the reactance ratio and/or impedance phase angle ratio may be used to determine epithelial coverage, and in some examples, the reactance ratio and/or impedance phase angle ratio may be multiplied by a calibration procedure. For example, a calibration procedure, curve, algorithm, data set, and the like, may be established by correlating a plurality of wound sites' histological epithelialization to wound bed reactances, e.g., across a population of patients. In some examples, the calibration procedure, curve, algorithm, data set, and the like may improve the accuracy of the reactance ratio and/or impedance phase angle ratio as a metric of epithelial coverage.

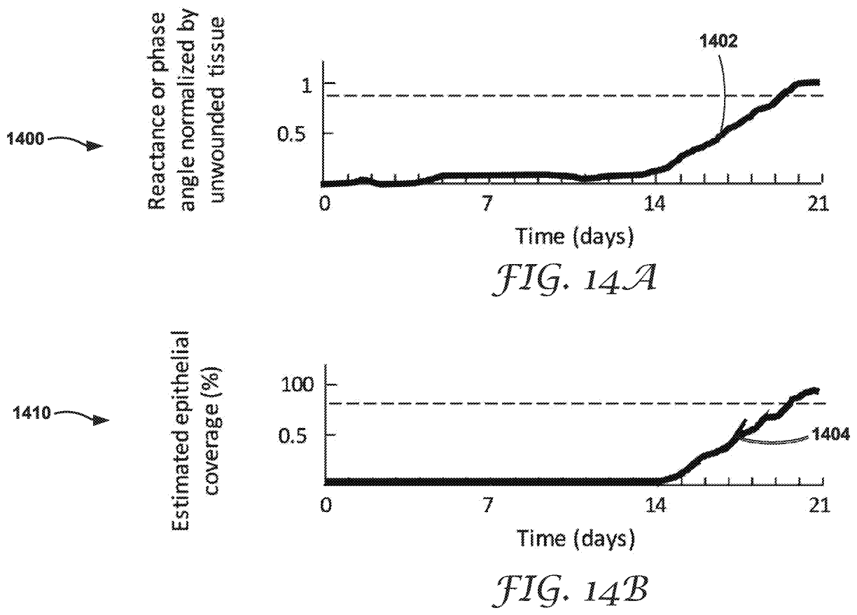

By way of example, FIG. 14A is a plot 1400 of example reactance ratios and/or impedance phase angle ratios measured at a predetermined frequency as a function of time, in accordance with the techniques described in this disclosure. FIG. 14B is a plot 1410 of an example corresponding epithelial coverage based on the reactance ratios and/or impedance phase angles of FIG. 14A as a function of time, in accordance with the techniques described in this disclosure. FIG. 14A illustrates a reactance and/or impedance phase angle ratio of wounded tissue site 150 to unwounded tissue site 152 as a function of time corresponding to impedance measurements 702-712 taken at six times after the occurrence of a wound at tissue site 150, e.g., days 0, 3, 7, 10, 14, and 16, illustrated and described above with reference to FIG. 7. Corresponding impedance measurements at tissue site 152 may be taken concurrently with impedance measurements 702-712, and processing circuitry may determine and store the ratios of reactances and/or impedance phase angles of each of the measurements. In the examples shown, the curve 1402 illustrates a plot of those ratios and the curve 1412 illustrates a plot of epithelial coverage percentage based on those ratios, e.g., either without calibrating or after having operated on curve 1402 via a calibration procedure, curve, algorithm, data set, and the like.

Processing circuitry 408 may determine that tissue site 150 is healed based on the determined epithelial coverage being greater than a predetermined threshold (1308). For example, processing circuitry 408 may determine that a wound is healed when curve 1402 exceeds 0.9 and/or curve 1412 exceeds 90%, as illustrated near day 20 as illustrated in FIGS. 14A and 14B.

The techniques described in this disclosure may be implemented, at least in part, in hardware, software, firmware, or any combination thereof. For example, various aspects of the described techniques may be implemented within one or more processors, including one or more microprocessors, digital signal processors (DSPs), application specific integrated circuits (ASICs), field programmable gate arrays (FPGAs), processing circuitry (e.g., fixed function circuitry, programmable circuitry, or any combination of fixed function circuitry and programmable circuitry), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit including hardware may also perform one or more of the techniques of this disclosure.

Such hardware, software, and firmware may be implemented within the same device or within separate devices to support the various techniques described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware, firmware, or software components. Rather, functionality associated with one or more modules or units may be performed by separate hardware, firmware, or software components, or integrated within common or separate hardware, firmware, or software components.

The techniques described in this disclosure may also be embodied or encoded in an article of manufacture including a computer-readable storage medium encoded with instructions. Instructions embedded or encoded in an article of manufacture including a computer-readable storage medium, may cause one or more programmable processors, or other processors, to implement one or more of the techniques described herein, such as when instructions included or encoded in the computer-readable storage medium are executed by the one or more processors. Computer readable storage media may include random access memory (RAM), read only memory (ROM), programmable read only memory (PROM), erasable programmable read only memory (EPROM), electronically erasable programmable read only memory (EEPROM), flash memory, a hard disk, a compact disc ROM (CD-ROM), a floppy disk, a cassette, magnetic media, optical media, or other computer readable media. In some examples, an article of manufacture may include one or more computer-readable storage media.

In some examples, a computer-readable storage medium may include a non-transitory medium. The term “non-transitory” may indicate that the storage medium is not embodied in a carrier wave or a propagated signal. In certain examples, a non-transitory storage medium may store data that can, over time, change (e.g., in RAM or cache).

The following examples may illustrate one or more aspects of the disclosure:

Example 1: A method includes applying an electrical signal to a tissue; measuring an impedance of the tissue based on the applied electrical signal; determining information indicative of a stage of wound healing based on the impedance; and outputting information indicative of the stage of wound healing.

Example 2: The method of example 1, wherein the electrical signal comprises a predetermined frequency.

Example 3: The method of any of examples 1-2, wherein the stage of wound healing comprises one of an inflammation stage, a proliferation stage, a remodeling stage, and a healed stage.

Example 4: The method of any of example 3, wherein determining information indicative of a proliferation stage comprises determining that the impedance comprises a resistance less than a first predetermined threshold value and a reactance greater than a second predetermined threshold value.

Example 5: The method of example 4, wherein determining information indicative of an inflammation stage comprises: determining that the proliferation stage has not occurred; and determining that the impedance comprises one of a resistance greater than the first predetermined threshold value or a negative reactance less than the second predetermined threshold value

Example 6: The method of any of examples 3-5, wherein determining a remodeling stage comprises: determining that the proliferation stage has occurred; and determining that the impedance comprises one of a resistance greater than the first predetermined threshold value or a negative reactance less than the second predetermined threshold value.

Example 7: The method of any of examples 3-6, wherein determining a healed stage comprises determining that the impedance is substantially equal to a baseline impedance measured of unwounded tissue.

Example 8: The method of any of examples 1-7, further includes applying a second electrical signal having the predetermined frequency to the tissue after applying the first electrical signal by a predetermined time interval; measuring a second impedance of the tissue based on the second applied electrical signal; and wherein determining information indicative of the stage of wound healing is based on the first and second impedances.

Example 9: The method of example 8, wherein determining information indicative of the stage of wound healing based on determining that the proliferation stage has not occurred after a predetermined number of time intervals have occurred.

Example 10: The method of example 8, wherein determining information indicative of the stage of healing based on the first and second impedances comprises determining a remodeling stage based on a rate of change of one of a reactance rate of change being less than a threshold reactance rate of change or an impedance phase angle rate of change being less than a threshold impedance phase angle rate of change between the first and second impedances.

Example 11: The method of any of examples 8-10 further comprising determining an amount of granulation tissue based on a resistance change between the first and second impedances.

Example 12: The method of any of examples 1-11, wherein the first device is configured to apply the electrical signal to the tissue via at least one of electrical contacts configured to be coupled to the tissue, capacitive coupling, or inductive coupling.

Example 13: A system includes a first device configured to apply an electrical signal to a tissue; and a second device configured to: measure an impedance of the tissue based on the applied electrical signal; determine information indicative of a stage of wound healing based on the impedance; and output information indicative of the stage of wound healing.

Example 14: The system of example 12, wherein the electrical signal comprises a predetermined frequency.

Example 15: The system of any of examples 13-14, wherein the first device is configured to apply the electrical signal to the tissue via at least one of electrical contacts configured to be coupled to the tissue or inductive coupling.

Example 16: The system of any of examples 13-15, wherein the stage of wound healing comprises one of an inflammation stage, a proliferation stage, a remodeling stage, and a healed stage.

Example 17: The system of example 16, wherein determining information indicative of a proliferation stage comprises determining that the impedance comprises a resistance less than a first predetermined threshold value and a reactance greater than a second predetermined threshold value, wherein determining information indicative of an inflammation stage comprises: determining that the proliferation stage has not occurred; and determining that the impedance comprises one of a resistance greater than the first predetermined threshold value or a negative reactance less than the second predetermined threshold value, wherein determining a remodeling stage comprises: determining that the proliferation stage has occurred; and determining that the impedance comprises one of a resistance greater than the first predetermined threshold value or a negative reactance less than the second predetermined threshold value, wherein determining a healed stage comprises determining that the impedance is substantially equal to a baseline impedance measured of unwounded tissue.

Example 18: The system of any of examples 13-17, wherein the first device is further configured to apply a second electrical signal having the predetermined frequency to the tissue after applying the first electrical signal by a predetermined time interval, wherein the second device is further configured to: measure a second impedance of the tissue based on the second applied electrical signal; and determine information indicative of the stage of wound healing is based on the first and second impedances.

Example 19: The system of example 18, wherein determining information indicative of the stage of wound healing based on the first and second impedances comprises determining a chronic wound stage based on determining that the proliferation stage has not occurred after a predetermined number of time intervals have occurred.

Example 20: The system of example 18, wherein determining information indicative of the stage of healing based on the first and second impedances comprises determining a remodeling stage based on a rate of change of one of a reactance rate of change being less than a threshold reactance rate of change or an impedance phase angle rate of change being less than a threshold impedance phase angle rate of change between the first and second impedances.

Example 21: The system of example 18-20 further comprising determining an amount of granulation tissue based on a resistance change between the first and second impedances.

Example 22: A dressing includes a first device configured to apply an electrical signal through wounded tissue at two or more predetermined time intervals via one of electrical contacts configured to be coupled to the tissue, capacitive coupling, or inductive coupling; and a second device configured to: measure an impedance of the wounded tissue based on the applied electrical signal at each of the two or more predetermined time intervals; determine a resistance and a reactance at each of the two or more predetermined time intervals based on the measured impedances; determine a stage of healing based on the determined resistances and reactances at each of the two or more predetermined time intervals; and output the stage of healing.

Example 23: A system comprising: a means for applying an electrical signal to a tissue; a means for measuring an impedance of the tissue based on the applied electrical signal; a means for determining information indicative of a stage of wound healing based on the impedance; and a means for outputting information indicative of the stage of wound healing.

Various examples have been described. These and other examples are within the scope of the following claims.

1. A system comprising:
a first device configured to apply an electrical signal to a tissue; and
a second device configured to:
measure an impedance of the tissue based on the applied electrical signal;
determine information indicative of a stage of wound healing based on the impedance; and
output information indicative of the stage of wound healing.

2. The system of claim 1, wherein the electrical signal comprises a predetermined frequency.

3. The system of claim 1, wherein the first device is configured to apply the electrical signal to the tissue via at least one of electrical contacts configured to be coupled to the tissue, capacitive coupling, or inductive coupling.

4. The system of any of claim 1, wherein the stage of wound healing comprises one of an inflammation stage, a proliferation stage, a remodeling stage, and a healed stage.

5. The system of claim 4, wherein to determine the information indicative of the proliferation stage, the second device is configured to determine that the impedance comprises a resistance less than a first predetermined threshold value and a reactance greater than a second predetermined threshold value,
wherein to determine the information indicative of the inflammation stage, the second device is configured to:
determine that the proliferation stage has not occurred; and
determine that the impedance comprises one of a resistance greater than the first predetermined threshold value or a negative reactance less than the second predetermined threshold value,
wherein to determine the remodeling stage, the second device is configured to:
determine that the proliferation stage has occurred; and
determine that the impedance comprises one of a resistance greater than the first predetermined threshold value or a negative reactance less than the second predetermined threshold value, and
wherein to determine the healed stage, the second device is configured to determine that the impedance is substantially equal to a baseline impedance measured of unwounded tissue.

6. The system of claim 1, wherein the first device is further configured to apply a second electrical signal having the predetermined frequency to the tissue after applying the first electrical signal by a predetermined time interval, wherein the second device is further configured to:
measure a second impedance of the tissue based on the second applied electrical signal; and
determine information indicative of the stage of wound healing is based on the first and second impedances.

7. The system of claim 6, wherein determining information indicative of the stage of wound healing based on the first and second impedances comprises determining a chronic wound stage based on determining that the proliferation stage has not occurred after a predetermined number of time intervals have occurred.

8. The system of claim 7, wherein to determine the information indicative of the stage of healing based on the first and second impedances, the second device is configured to determine a remodeling stage based on a rate of change of one of a reactance rate of change being less than a threshold reactance rate of change or an impedance phase angle rate of change being less than a threshold impedance phase angle rate of change between the first and second impedances.

9. The system of claim 6, wherein the second device is further configured to determine an amount of granulation tissue based on a resistance change between the first and second impedances.

10. A method comprising:
applying an electrical signal to a tissue;
measuring an impedance of the tissue based on the applied electrical signal;
determining information indicative of a stage of wound healing based on the impedance; and
outputting information indicative of the stage of wound healing.

11. The method of claim 10, wherein the stage of wound healing comprises one of an inflammation stage, a proliferation stage, a remodeling stage, and a healed stage.

12. The method of claim 11, wherein determining the information indicative of the proliferation stage comprises determining that the impedance comprises a resistance less than a first predetermined threshold value and a reactance greater than a second predetermined threshold value.

13. The method of claim 12, wherein determining the information indicative of the inflammation stage comprises:
determining that the proliferation stage has not occurred; and
determining that the impedance comprises one of a resistance greater than the first predetermined threshold value or a negative reactance less than the second predetermined threshold value.

14. The method of claim 13, further comprising applying the electrical signal to the tissue via at least one of electrical contacts configured to be coupled to the tissue, capacitive coupling, or inductive coupling.

15. A dressing comprising:
a first device configured to apply an electrical signal through wounded tissue at two or more predetermined time intervals via one of electrical contacts configured to be coupled to the tissue, capacitive coupling, or inductive coupling; and
a second device configured to:
measure an impedance of the wounded tissue based on the applied electrical signal at each of the two or more predetermined time intervals;
determine a resistance and a reactance at each of the two or more predetermined time intervals based on the measured impedances;
determine a stage of healing based on the determined resistances and reactances at each of the two or more predetermined time intervals; and
output the stage of healing.
